(19) United States
(12) Patent Application Publication
(10) Pub. No.: US 2006/0140867 A1
(43) Pub. Date: Jun. 29, 2006

(54) Coated stent assembly and coating materials

(76) Inventors: Jeffrey L. Helfer, Victor, NY (US); Xingwu Wang, Wellsville, NY (US); Robert W. Gray, Rochester, NY (US); Howard J. Greenwald, Rochester, NY (US)

Correspondence Address: HOWARD J. GREENWALD P.C., 349 W. COMMERCIAL STREET SUITE 2490, EAST ROCHESTER, NY, 14445-2408, US

(21) Appl. No.: 11/128,591

(22) Filed: May 13, 2005

Related U.S. Application Data
(63) Continuation-in-part of application No. 11/023,873, filed on Dec. 28, 2004, now abandoned.

(51) Int. Cl. A61K 49/06

(52) U.S. Cl. 424/9.32; 977/930

(57) ABSTRACT

A high magnetic susceptibility nanomagnetic material that may be attached to recognition molecules and other therapeutic biological materials so as to be targeted to specific biologic tissues, thereby enabling the presence of the targeted tissue to be detected under magnetic resonance imaging with much greater sensitivity. Also a stent coated with such nanomagnetic material to enable artifact free imaging of such stent under magnetic resonance imaging.

### CROSS-REFERENCE TO RELATED PATENT APPLICATIONS

This application is a continuation-in-part of applicants' U.S. patent application Ser. No. 11/023,873 filed on Dec. 28, 2004.

### FIELD OF THE INVENTION

A contrast agent assembly adapted to be used within a patient during magnetic resonance imaging (MRI) analyses. In one embodiment, the contrast agent assembly is comprised of a recognition molecule attached to or contiguous with nanomagnetic particles.

### BACKGROUND OF THE INVENTION

Magnetic Resonance Imaging (“MRI”) is rapidly becoming a dominant radiological imaging method due to such advantages as superb soft tissue contrast; no ionizing radiation; images that are not obstructed by bone; multi-plane images without the need to reposition a patient; tissue function analysis capabilities; and MRI-guided surgery.

MR imaging places a patient within the bore of a powerful magnet and passes radio waves through the patient's body in a particular sequence of very short pulses. Each pulse causes a responding pulse of radio waves to be emitted from the patient's tissues. The location from which the signals have originated is recorded by a computer, which then produces a two-dimensional picture representing a predetermined section or slice of the patient.

Different body tissues emit characteristic MR signals which determine whether they will appear white, gray, or black in the image. Tissues that emit strong MR signals appear white in MR images, whereas those emitting little or no signal appear black.

The strength of the MR signal depends upon the collective, or net, magnetic effect of the large number of atomic nuclei within a specific volume of tissue (called a “voxel).” If a tissue voxel contains more nuclei aligned in one direction (via the externally applied magnetic field) than in other directions, the tissue will be temporarily magnetized in that particular direction.

The maximum magnetization that can be produced depends upon three factors: (1) the concentration (density) of magnetic nuclei in the tissue sample, (2) the magnetic sensitivity of the nuclei (i.e. their ability to be magnetized), and (3) the strength of the externally applied magnetic field. The amount of tissue magnetization determines the strength of the RF signals emitted by the tissue during an imaging or analytical procedure. This, in turn, affects image quality and imaging time requirements.

The ability to image tissues, particularly small soft tissue masses, can at times be limited by either the very weak MRI signals created by the tissues or insufficient difference in the MRI signal of the tissues relative to the MRI signals received from surrounding tissues (often referred to as “MRI contrast”).

The present invention, in one embodiment thereof, provides the means to further enhance MRI soft tissue visualization capability.

### SUMMARY OF THE INVENTION

The magnetic strength of a compound can be described in terms of its ability to be magnetized, commonly referred to as its magnetic susceptibility. Materials with high magnetic susceptibility have a high Electro-Magnetic Unit density (“EMU”) per unit volume. Ferromagnetic materials, such as iron, have very high magnetic susceptibility and very high EMUs per unit volume, whereas tissues that produce very weak magnetic signals have a very low magnetic susceptibilities and very low EMU per unit volume. Other magnetic materials, such as gadolinium, dysprosium, or nickel, have EMUs that are stronger than biological tissues, but still much weaker than iron ferromagnetic materials.

The EMUs of iron are not durable, i.e., iron is very reactive and reacts, e.g., with oxygen to form compounds with lower EMU's .

The present invention, in one embodiment thereof, delivers nano-meter sized particles of high magnetic susceptibility materials, such as ferromagnetic materials (i.e. materials that produce very high magnetic signals or very high EMUs) to tissues to be imaged to improve MR visualization of these tissues. The nano-meter sized particles of this embodiment are not reactive with oxygen and, thus, maintain their magnetic strengths over time.

In another embodiment of the invention, nano-magnetic particles are fabricated into a mass of one or more particles which are attached to a tissue recognition molecule (such as an antibody) which has an affinity for a particular type of tissue (such as a particular type of cancer cell). These nano-magnetic particle/antibody masses are then delivered into the body (e.g. circulatory system, lymph system, stomach, etc.) to allow them to come into contact with and become immobilized to the target tissue (i.e. cancer cell). The very high magnetic signal created by the nano-magnetic particles in one aspect of this invention creates a very high magnetic signal at the site of the targeted tissue, thereby enabling the presence of the targeted tissue to be detected under MR imaging with much greater sensitivity.

In another embodiment, nano-magnetic particles are attached to multiple recognition molecules, such as antibodies, with affinities for different tissue types, and delivered into the body, thus providing the ability to detect the presence of multiple tissue types, such as multiple cancer types.

In yet another embodiment, nano-magnetic particles are attached to multiple recognition molecules, such as antibodies, with affinities for the same tissue types, and delivered into the body, thus providing the ability to detect the presence of a specific tissue type with much greater specificity.

In yet another embodiment, nano-magnetic particles are attached to recognition molecules, such as antibodies, that have affinities for metabolic agents, such as enzymes or proteins, and delivered into the body, thus providing the ability to detect the presence of the targeted metabolic agent. In one aspect of this embodiment, e.g., nano-magnetic particles are attached to recognition molecules with affinities for CK-MB, an enzyme, or Troponin, a protein, materials whose concentrations change in response to damage to cardiac muscle, thereby providing the means to detect with greater sensitivity the incidence of a heart attack, as well as the magnitude and location of the damaged heart muscle.

In yet another embodiment, nano-magnetic particles are attached to materials, such as food stuffs or other ingestible agents, that are known to be preferentially absorbed by tissues to be imaged (e.g. nano-magnetic particles are attached to beta carotene which is known to be preferentially absorbed by arterial stenosis) and delivered into the body, thus providing the ability to detect the presence of a specific tissue type(s) with much greater specificity (e.g. the presence of an arterial stenosis in this example).

In yet another embodiment, nano-magnetic particles are attached to therapeutic agents, such as drugs, where the intent is that the drug is to be preferentially absorbed by tissues to be treated. For example, in one aspect of this embodiment, nano-magnetic particles are attached to a chemo-toxin designed to destroy cancer cells, thus providing the ability to detect the ability of the drug to reach and enter into the target tissues, the cancer tumor in this example.

In yet another embodiment, nano-magnetic particles are attached to a combination of recognition molecule(s) or preferentially absorbed materials (as above) as well as a chemo-attractant—a material know to attract other chemical or biochemical agents such as chemo-toxins, thereby providing the ability to detect the extent to which chemo-attractants have reached targeted tissues (e.g. cancer tumors) and therefore the likelihood that the targeted tissue will be exposed to the desired chemical or biochemical agent (e.g. chemo-toxin) and therefore the effectiveness of the proposed chemical or biochemical therapeutic agent.

In yet another embodiment, nano-magnetic particles are attached to inhaled agents, such as micro-spheres, which are inhaled into the respiratory system, thereby providing the ability to detect with greater sensitivity the active geometry of the respiratory system, including the presence and extent of respiratory diseases known to occlude the airways (e.g. pneumonia or bronchitis).

In yet another embodiment, nano-magnetic particles are attached to devices placed into the body to enable the presence and physical characteristics of the device to be detected with greater sensitivity.

In yet another embodiment, nano-magnetic particles are fabricated into masses in which the magnetization vectors of each nano-magnetic particle are aligned in approximately the same directions so as to produce a very strong magnetic signal.

In yet another embodiment, nano-magnetic particles are fabricated into masses in which the magnetization vectors of each nano-magnetic particle are deliberately misaligned so as to produce a modulated magnetic signal (i.e. a signal whose strength is lower than when all nano-particle magnetization vectors are aligned).

In yet another embodiment, nano-magnetic particles are fabricated into masses containing a varying number of nano-magnetic particles so as to modulate the strength of the magnetic signal generated by each mass of nano-magnetic particles (i.e. greater numbers of particles produce a stronger magnetic signal).

In yet another embodiment, nano-magnetic particles of different materials having varying degrees of magnetic susceptibility or EMU per unit volume (e.g. iron versus nickel) are utilized so as to modulate the strength of the magnetic signal generated by each mass of nano-magnetic particles (i.e. materials with greater magnetic susceptibility and EMU per unit volume produce a stronger magnetic signal).

In a further embodiment, nanomagnetic particles having varying electromagnetic properties may be bound to biochemical binding agents in a manner that permits an MRI system to differentiate tissue types, disease states, or other diagnostic metrics.

In yet another embodiment, nano-magnetic particles are sealed with non-biodegradable materials to prevent them from coming into direct contact with body tissues.

In yet another embodiment, nano-magnetic particles are sealed with dissolvable materials to provide time-release capability.

### BRIEF DESCRIPTION OF SOME OF THE DRAWINGS

The invention will be described by reference to the following drawings, in which like numerals refer to like elements, and in which:

FIG. 1 is original raw magnitude data from a magnetic resonance imaging experiment with an embodiment of the invention;

FIG. 2 is phase image data from the same experiment as in FIG. 1;

FIG. 3 illustrates the results as a result of an edge-tracing mathematical calculation performed on the data of FIG. 2;

FIG. 4 is a schematic representation of a multilayer embodiment of the invention;

FIG. 4A is a simplified circuit model representation of the multilayer embodiment of FIG. 4;

FIG. 5 is a perspective view of a coated stent embodiment of the invention;

FIG. 6 is a perspective view of another coated stent embodiment of the invention;

FIG. 7 is a flow diagram of a process for optimizing the coated stent embodiments of the invention;

FIG. 8 is an image produced from a number of different coatings on a series of copper stents;

FIG. 9 is an image produced from the image of FIG. 8, wherein the phase was equalized;

FIG. 10 is an image produced from a series of copper rings that were used to simulate the stents of FIG. 8, wherein only the magnitude was equalized; and

FIG. 11 is an image produced from the image of FIG. 10, wherein the phase has been equalized.

### DETAILED DESCRIPTION OF THE INVENTION

Other MR imaging contrasts agents have been used in the past, but without the advantages of contrast agents of the present invention.

By way of illustration, the elements Gadolinium and Dysprosium have been used to coat medical devices to improve the visualization of the device under MR imaging. However, the magnetic susceptibilities of these materials are 755,000 and 103,500×10⁻⁶ cgs respectively, whereas iron is 1,000,000 or more; see, e.g., the CRC Handbook of Chemistry and Physics, College Edition, 50^{th} edition, 1969-70, page E-130.

The magnetic susceptibility of iron is greater than that of Gadolinium and Dysprosium. At a temperature of 300 degrees Kelvin, the EMU per unit volume of an iron nano-magnetic particle mass will be much higher than that of an equivalent volume of a suspension of Gadolinium and Dysprosium. This means that a stronger magnetic signal, hence greater image quality, can be achieved with a much smaller mass of iron nano-magnetic particles. Smaller mass is very important when, for example, contrast agents must be attached to very small recognition molecules, inhalation particles, drug molecules, or devices whose physical properties (e.g. physical size or flexibility) cannot be altered.

Reference may be had, e.g., to a text by R. S. Tebble et al. entitled “Magnetic Materials” published by Wiley-Interscience (New York, N.Y., 1969). In Table 2.1b of such text (see page 51), it will be seen that the iron zero-degree Kelvin saturation magnetization is 1752 e.m.u/cubic centimeter, which equates to a saturation magnetization of 221.7 e.m.u. per gram. Iron also has a Curie temperature of 770 degrees Celsius, which equates to Curie temperature of 1043 degrees Kelvin.

Reference also may be had to page 197 of the Tebble text. Referring to this page, it will be seen that gadolinium has a 0 degree Kelvin saturation magnetization of 268 e.m.u/gram, that equates to 1950 emu per cubic centimeter. However, it should be noted that gadolinium has a Curie temperature of 293.2 degrees Kelvin. Thus as will be apparent, at room temperature the magnetic property of gadolinium is substantially weaker than the magnetic property of iron.

To the best of applicants' knowledge and belief, no one in the prior art has provided FeAlN nanoparticles as contrast agents. Applicants have discovered that, unexpectedly, applicants' nitrogen-containing nanoparticles substantially retain the desirable magnetic properties of pure iron nanoparticles while, when doped with nitrogen, provides insulating properties; this is unexpected, for other iron compounds have substantially weaker magnetic properties than pure iron.

Additionally, the coatings of this invention can be constructed to have both insulating and conductive properties and to exhibit both inductive reactance and capacitative reactance in an MRI field. This tunable nature of such coatings, and the insulating properties of such coatings, allows one to adjust the extent to which eddy currents flow on the surface of a coated conductor when subjected to MRI radiation. By comparison, the pure iron nanoparticles, in addition to providing comparable magnetic properties, do not provide insulating properties and are not tunable. Thus, e.g., the pure iron particles will create a substantial amount of inductive reactance in an MRI RF field, and such large net reactance will produce image artifacts. The coatings of this invention, by comparison, can be tuned to have little or no net reactance and, thus, little or no image artifacts in an MRI RF field.

Furthermore, iron nanoparticles are not a stable in many environments, readily combining with oxygen to form ferrites that have substantially inferior magnetic properties to the pure iron particles. By comparison, the FeAlN particles are stable in an oxygen-containing environment and retain their magnetic properties when exposed to oxygen.

The FeAlN nanoparticles of this invention also have substantially different properties than Dysprosium contrast agents. At a temperature of zero degrees Kelvin, the Dysprosium contrast agents have a saturation magnetization of 350 e.m.u. per gram; however, its Cure temperature is 85 degrees Kelvin. As will be apparent, at room temperature the Dysprosium contrast agents are not ferromagnetic.

The FeAlN nanoparticles of this invention have substantially different properties than nickel contrast agents, which are toxic and, when exposed to oxygen, form nickel oxides, thereby destroying the magnetic properties of the pure nickel. It should be noted, and referring to page 51 of the Tebble text, that nickel has a saturation magnetization of 510 e.m.u/cubic centimeter, substantially worse than iron's 1752 e.m.u. per cubic centimeter.

Another benefit of the present invention is the ability to modify the composition of the magnetic particle mass, such as by varying the type or number of ferromagnetic or superparamagnetic particles, or the magnetic orientation of the magnetic particles, to enable the magnetic signal to be modulated. This means that different particles can be designed to provide different signals so as to, for example, control signal intensity so as to better differentiate between the magnetic signals received from nano-magnetic particles attached to targeted tissues and their surrounding tissues (e.g. better differentiate between the targeted tissues and fat, brain matter, blood, etc.) or to differentiate between different nano-magnetic particle types.

Yet another benefit is the ability to achieve higher concentrations of ferromagnetic materials (such as iron) relative to Gadolinium and Dysprosium, thus achieving higher EMUs per unit volume and higher relative magnetic susceptibility.

Yet another benefit is low risk potential due to low toxicity, provided by both the benign nature of the nano-magnetic particles (i.e. iron is already present in the body) and the very low concentration of nano-magnetic particles used (due to their much higher magnetic susceptibility and EMU per unit volume). In contrast, other MRI contrast agents, such as Gadolinium and Dysprosium, are known to react with water and are soluble in very weak acids, such as those found in the body.

Yet another benefit is the ability to reduce image acquisition time, due to the much higher magnetic signal produced by the nano-magnetic particles.

Yet another benefit is the ease of manufacturing and low manufacturing cost to produce the nano-magnetic particles, including lower cost of the raw materials.

Yet another benefit is the relatively short time to market, which is enabled due to existing use of such materials and small particles within the body.

Recognition Molecules

In one embodiment of the invention, a recognition molecule is used in conjunction with magnetic particles such as, e.g., nanomagnetic particles.

As is known to those skilled in the art, recognition is a specific binding interaction occurring between macromolecules, as that between an immunocyte and an antigen. See, e.g., page 404 of J. Stensch's “Dictionary of Biochemistry and Molecular Biology,” Second Edition (John Wiley & Sons, New York, N.Y., 1989).

As is also known to those skilled in the art, a receptor is a target site at the molecular level to which a substance becomes bound as a result of a specific interaction; see, e.g., page 404 of the Stensch dictionary.

A recognition molecule is a moiety that is adapted to, e.g., bind to a particular receptor. These recognition molecules are well known to those in the art.

By way of illustration, U.S. Pat. No. 4,652,532 discloses a biochemical method of assaying for ligand molecules in fluids based upon the specific interaction of a ligand and a ligand-recognition molecule that binds the ligand; the entire disclosure of this United States patent is hereby incorporated by reference into this specification.

A ligand is an atom, a group of atoms, or a molecule that binds to a macromolecule; see, e.g., page 273 of J. Stensch's “Dictionary of Biochemistry and Molecular Biology,” Second Edition (John Wiley & Sons, New York, N.Y., 1989). As so defined in this dictionary, a ligand is comprehended within the term recognition molecule, as that term is used in this specification.

A ligand has also been defined as “Any molecule that binds to a specific site on a protein or other molecule . . . ;” see, e.g., page G-14 of the Glossary of Bruce Alberts et al.'s “Molecular Biology of The Cell,” Third Edition (Garland Publishing, New York, N.Y., 1994). As so defined in this dictionary, a ligand is comprehended within the term recognition molecule, as that term is used in this specification.

In U.S. Pat. No. 4,652,532, at lines 43 et seq. of column 2, it is disclosed that: “Ligands are typically, but not necessarily, small molecular weight molecules such as drugs, steroid hormones, and other bioactive molecules. Ligand-recognition molecules are generally but also not necessarily large molecular weight molecules, usually proteins such as antibody. Both can be isolated by following biochemical isolation and purification protocols that are characteristically unique for a specific substance, or, in some instances, they can be purchased commercially.” As will be apparent, because both the “ . . . large molecular weight molecules . . . ” and the “ . . . small molecular weight molecules . . . ” “recognize” each other, they are both a recognition molecule, as that term is used in this specification.

One specific recognition molecule discussed in U.S. Pat. No. 4,652,532 is an antigen. As is known to those skilled in the art, and referring to page 31 of the Stensch dictionary, an antigen is “A substance, frequently a protein, that can stimulate an animal organism to produce antibodies and that can combine specifically with the antibodies thus produced . . . . ” These antigens are discussed at lines 53 et seq. of United States patent, wherein it is disclosed that: “Antigen and antibody are preferred embodiments of a ligand and ligand-recognition molecules respectively. Antibodies with exquisite antigenic specificity can be produced by immunization of animals with antigen, either alone or with adjuvant. For poor antigenic substances, particularly steroids or small peptides, in addition to injecting adjuvant, it is often necessary to couple these ligands to an antigenic carrier . . . .”

Another specific recognition molecule discussed in U.S. Pat. No. 4,652,532 is an antibody. As is disclosed on page 30 of the Stensch dictionary, an antibody is “A glycoprotein of the globulin type that is formed in an animal organism is response to the administration of an antigen and that is capable of combining specifically with that antigen.

Thus, e.g., an immunoglobulin is a recognition molecule, as that term is used in this specification. Referring to page 236 of the Stensch dictionary, an immunoglobulin is: “1. A protein of animal origin that has a known antibody activity. 2. A protein that is closely related to an antibody by its chemical structure and by its antigenic specificity . . . .”

In one embodiment of this invention, a complex assembly is formed comprised of magnetic material and two or more recognition molecules bound to each other. U.S. Pat. No. 4,652,532 discloses assemblies of two or more recognition molecules bound to each other. Thus, e.g., in “EXAMPLE 1” it describes the preparation of a “LIGAND/LIGAND RECOGNITION MOLECULE COMPLEX FORMATION.” The process for making such a preparation is partially described in claim 1 of the patent which describes, in relevant part: “ . . . combining said ligand with a molecule recognizing said ligand to form a ligand recognition molecule, wherein said ligand or said ligand recognition molecule is reactive to become a free radical . . . .”

By way of further illustration, U.S. Pat. No. 5,458,878 discloses: “Multifunctional, recombinant cytotoxic fusion proteins containing at least two different recognition molecules . . . for killing cells expressing receptors to which the recognition molecules bind with specificity . . . ;” the entire disclosure of this United States patent application is hereby incorporated by reference into this specification.

Reference also may be had, e.g., to related U.S. Pat. No. 5,705,163, the entire disclosure of which is also incorporated by reference into this specification.

U.S. Pat. No. 5,458,878 describes and claims: “A fusion protein comprising a recombinant Pseudomonas exotoxin (PE) molecule, a first recognition moiety for binding a target cell, and a carboxyl terminal sequence of 4 to 16 residues which permits translocation of said fusion protein into the target cell cytosol, the first recognition moiety being inserted in domain III of PE after residue 600 and before residue 613.” As is disclosed in column 1 of such patent: “The present invention is related generally to the making of improved recombinant immunotoxins. More particularly, the present invention is related to the construction of a recombinant Pseudomonas exotoxin (rPE) with specific cloning sites for the insertion of recognition molecules at least at the carboxyl end of the PE to achieve target-directed cytotoxicity and for the construction of recombinant multifunctional chimetic cytotoxic proteins.”

By way of further illustration, U.S. Pat. No. 5,482,836 describes several “molecular recognition systems,” such as “ . . . an antigen/antibody, an avidin/biotin, a streptavidin/biotin, a protein A/Ig and a lectin/carbohydrate system . . . ;” the entire disclosure of this United States patent is hereby incorporated by reference into this specification.

The term “molecular recognition system,” as used in U.S. Pat. No. 5,482,836 (and also as used in this specification) is “ . . . a system of at least two molecules which have a high capacity of molecular recognition for each other and a high capacity to specifically bind to each other . . . ” (see lines 48-51 of column 6 of this patent). As is specifically disclosed in this column 6: A “molecular recognition system” is a system of at least two molecules which have a high capacity of molecular recognition for each other and a high capacity to specifically bind to each other. Molecular recognition systems for use in the invention are conventional and are not described here in detail. Techniques for preparing and utilizing such systems are well-known in the literature and are exemplified in the publication Tijssen, P., Laboratory Techniques in Biochemistry and Molecular Biology Practice and Theories of Enzyme Immunoassays, (1988), eds. Burdon and Knippenberg, New York, Elsevier.”

As is also described in column 7 of U.S. Pat. No. 5,482,836: “Acceptable molecular recognition systems for use in the present invention include but are not limited to an antigen/antibody, an avidin/biotin, a streptavidin/biotin, a protein A/Ig and a lectin/carbohydrate system. The preferred embodiment of the invention uses the streptavidin/biotin molecular recognition system and the preferred oligonucleotide is a 5′-biotinylated homopyrimidine oligonucleotide. To form the intermolecular triple-helices, the sample containing the DNA is incubated in an acidic buffer with the biotinylated nucleotide. A mildly acidic buffer of pH 4.5-5.5 is preferred but acidic buffers ranging from a pH of about 3.5 to about 6.5 are acceptable. The preferred buffer is a sodium acetate/acetic acid buffer but other buffers such as sodium/citrate/citric acid, PIPES and sodium phosphate may also be used. With reactions at high pHs (6.0 or above), sodium phosphate buffer is used instead of sodium acetate/acetic acid buffer. The reaction medium containing the triple-helices is then incubated with the solid carrier fixed with the second recognition molecule of the molecular recognition system. The solid phase is preferably suspended in the same buffer as the buffer used to induce triple-helix formation. Again, the second recognition molecule must be a recognition molecule with higher affinity for the recognition molecule coupled to the oligonucleotide. When the recognition molecule coupled to the oligonucleotide is biotin, a preferred solid phase is a streptavidin coated solid phase. If the first recognition molecule coupled to the oligonucleotide is streptavidin, then the preferred second recognition molecule attached to the solid phase would be a biotin. When appropriate, the recognition molecules may be directly or indirectly coupled to the oligonucleotide or solid phase. However, if the recognition molecules are indirectly attached to the oligonucleotide, the problems of steric hindrance should be considered. For example, if streptavidin or avidin are chemically attached to oligonucleotides via linkers, care must be taken with respect to the length of the linker . . . . An example of an acceptable molecular recognition systems other than streptavidin/biotin system that may be used with the TAC method of the invention is the antigen/antibody system. An appropriate example of this system is the digoxigenin antigen and an anti-digoxigenin antibody system. (See, Current Protocol in Molecular Biology, (Eds. Ausbel, F. M., et al.), Supl. 12, Greene Publishing Associates and Wiley-Interscience, 1990.).”

By way of further illustration, U.S. Pat. No. 6,046,008 describes “ . . . a biological recognition molecule which specifically binds to said analyte and an analog thereof . . . ;” the entire disclosure of this United States patent is hereby incorporated by reference into this specification. This biological recognition molecule is used in an “IMMUNOLOLIGICALLY BASED STRIP TEST UTILIZING IONOPHORE MEMBRANES.” In particular, and as is described in the “ABSTRACT” of this patent, there is provided: “A testing apparatus 10 having an absorbent matrix 12, including a membrane 14 which contains a plurality of counter-ions 16. Chromoionophore (or fluorionophore)s 18 and affinophores 22 compete to carry ions into the membrane 14 and neutralize the charge of the counter-ions 16. Biological recognition molecules 42 bind to a portion of the affinophores 22 and prevent them from entering the membrane 14, thereby allowing more chromoionophore (or fluorionophore)s 18 to enter the membrane 14. The portion of affinophores 22 bound to the biological recognition molecules 42 is inversely proportional to the amount or concentration of analyte 40 occurring within the solution or medium 30. The result of this is that the color of the membrane-covered matrix changes in a manner related to the concentration of the analyte.”

Referring to the claims of U.S. Pat. No. 6,046,008, the biological recognition molecule used may be “ . . . an antibody . . . ” (claim 2), “ . . . a portion of a whole antibody containing a binding site . . . ” (claim 3), “ . . . a biological receptor for the analyte . . . ” (claim 4), or “ . . . a portion of a specific nucleotide having an affinity for the analyte . . . ” (claim 5).

By way of further illustration, U.S. Pat. No. 6,214,790 discloses “ . . . a serotonin recognition molecule . . . ;” the entire disclosure of this United States patent is hereby incorporated by reference into this specification.

U.S. Pat. No. 6,214,790 discusses, in relevant part, serotonin receptors and transporters. In column 1 of this patent, it is disclosed that: “Serotonin (5HT) is a neurotransmitter that is essential to brain function. Multiple serotonin receptors and transporters have been identified and cloned. Briefly, de novo synthesis of serotonin from tryptophan occurs in the cytoplasm of a cell. Once synthesized, vesicular monoamine transporters package the transmitter into vesicular compartments so that its release can be regulated. Once released into the synapse upon proper stimulation, the transmitter can bind specific serotonin receptors, can be degraded by specific enzymes, and/or can be transported back into a cell by specific plasma membrane serotonin transporters and then re-packaged into vesicles. Thus, both serotonin receptors and transporters specifically recognize serotonin.” As will be apparent, both serotonin receptors, serotonin transporters, and serotonin are “recognition molecules,” as that term is used in this specification.

By way of further illustration, published United States patent application U.S. 2002/0022266 discloses a drug delivery process in which: “a chemical or biological entity having a recognition molecule attached thereto or expressed thereby is introduced to the surface . . . .” In particular, this published patent application claims: “A method for delivery of a chemical or biological entity to a tissue or cellular surface comprising: binding a molecule to said surface, wherein said molecule comprises at least one reactive group that reacts with groups present on said surface, and at least one signaling molecule; and attaching said entity to said signaling molecule by means of a recognition molecule, wherein said recognition molecule is specific for said signaling molecule.”

Published United States patent application 2002/0022266 discloses that both “signaling molecules” and “recognition molecules” are “recognition molecules” as that term is used in this specification; i.e., because of their stereochemistry and/or their physical and/or chemical properties, they “recognize” each other. Thus, e.g., as is disclosed in page 2 of this published patent application: “The molecules of the present invention also include a “signaling molecule,” that can be specifically recognized by the recognition molecule attached to or expressed by the entity to be delivered to the target surface. This signaling molecule should therefore be selected in conjunction with the recognition molecule. Any group that will function as a signaling molecule is within the scope of the present invention, absent compatibility problems. The chemical or biological entity to be delivered is modified, if necessary, to include a molecule or other moiety (“recognition molecule”) that will recognize and bind with the signaling molecule. For example, the entity can be chemically modified through the attachment of a recognition molecule to its surface; such attachment can be effected by any means of attachment known in the art or organic or biochemistry. Alternatively, the entity can be genetically modified so as to express the recognition molecule. A preferred example of a suitable signaling molecule/recognition molecule combination is biotin and avidin. The biotin-avidin system for targeting is well-known to those skilled in the art. Suitable biotin is commercially available from Pierce as sulfo-NHS-Biotin MW 443.43 and sulfo-NHS-LC-LC-Biotin MW 669.75, and from Shearwater Polymers as NHS-PEG-Biotin MW approximately 3,400. Other suitable signaling molecule/recognition molecule combinations include ligands/receptors; antibody/antigen; primary antibody/secondary antibody; protein A/fc region of human immunoglobulin (IgGl); and protein C/fc region of IgGl. Several of these systems suitable for use in the current methods are commercially available and can be obtained from Pierce, Sigma and Molecular Probes.”

Published United States patent application US 2002/0022266 discloses how a particular cell type can be modified to have recognition molecules (“receptors”) incorporated into or onto the cell. Thus, as is disclosed at page 3 of this published patent application: “Similarly, the delivery of an appropriate cell type or a genetically modified cell to a treated region may be accomplished according to the present methods. Such delivery is affected by appropriately selecting the signaling molecule to be recognized by the desired cell type. The desired cell type can also be modified in vitro to provide surface receptors that will recognize the applied target. This can be done, for example, through chemical modification by physical attachment of the recognition molecule to the cell surface, or by genetic modification in which the cell is genetically engineered to express the recognition molecule. Any cell that it would be desirable to deliver to a patient is suitable for use in the present invention. Examples include stem cells and endothelial cells. Autologous and non-autologous cells can be used, such as mammalian cells with surface expressed protein. The surface expressed protein can be one needed by the patient, such as a protein that the patient cannot manufacture in sufficient quantity himself.”

The process described in published United States patent application US 2002/0022266 is claimed in claim 1 of the case, which describes a: “ . . . method for delivery of a chemical or biological entity to a tissue or cellular surface comprising: binding a molecule to said surface, wherein said molecule comprises at least one reactive group that reacts with groups present on said surface, and at least one signaling molecule; and attaching said entity to said signaling molecule by means of a recognition molecule, wherein said recognition molecule is specific for said signaling molecule.”

By way of further illustration, a tracer may be a “recognition molecule,” as that term is used in this specification. As is disclosed at page 488 of such Stensch dictionary, a tracer is: “1, An isotope, either radioactive or stable, that is used to label a compound. 2. A compound labeled with either a radioactive or a stable isotope.”

Published United States patent application 2002/0142484 (the entire disclosure of which is hereby incorporated by reference into this specification) discloses “ . . . novel caanabinol-based tracers suitable for use in immunoassays that detect cannabinoids in a biological sample. As is disclosed on page 1 of this published patent application: “Marijuana, a known psychoactive drug, is derived from plants of the hemp family that produce significant amounts of cannabinoids. In particular, the most important cannabinoid is Δ9-tetrahydrocannabinol (Δ9-THC), the major physiologically active constituent of marijuana. Δ9-THC is a controlled substance because it has both sedative and depressant-like effects on the cardiovascular and central nervous systems, as opposed to cannabidiol, a non-psychoactive constituent of marijuana. Through smoking marijuana, Δ9-THC is rapidly absorbed from the lungs into the blood stream and metabolized through 11-nor-Δ9-THC to a series of polar metabolites with 11-nor-Δ9-THC-carboxylic acid as the primary metabolite. Due to the common abuse of cannabinoids, there is a growing need for non-invasive and rapid tests to detect the presence of these controlled drugs in biological specimens . . . . To detect Δ9-THC using an immunoassay or immunosensor, a tracer molecule is usually used to compete with Δ9-THC or its metabolites. The tracer molecule is usually a labeled antigen or ligand, capable of binding to the same antigen or ligand binding site(s) of an antibody or receptor to Δ9-THC or its metabolites. In detecting controlled substances, most immunoassays have generally used the labeled illicit drugs themselves, (e.g., labeled Δ9-THC) as tracers to detect the presence and/or to quantify the analytes in the sample.

Recent use of non-controlled substances as starting materials in Δ9-THC tracers synthesis has also been reported by Wang, et al, in U.S. Pat. No. 5,264,373 entitled Fluorescence polarization Immunoassay for tetrahydrocannabinoids. In this patent, Wang discloses the use of fluorescein to label THC-analog based derivatives for use in a fluorescence polarization immunoassay.”

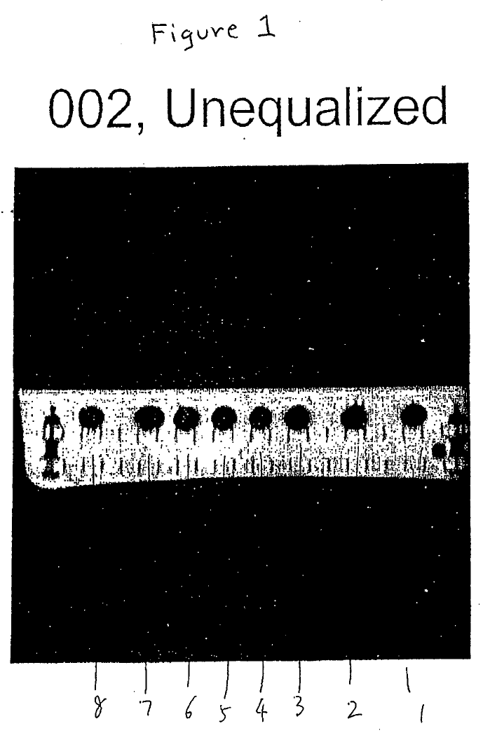

Published United States patent application US 2002/014284 then describes various “tracer recognition molecules” and their synthesis, stating that: (at page 1 thereof): “FIG. 1 generally depicts the various methods for synthesizing tracers used in the detection of Δ9-THC or it metabolites. Panel A, for example, depicts one of the common methods that use controlled substances, such as the illicit drug, 9-carboxy (or aldehyde)-Δ9-THC, as starting materials. These starting materials are coupled with labels at the carboxyl group attached to the carbon at position 9 on Δ9-THC to yield drug-based tracers. Panel B depicts an alternative method of synthesizing a tracer, which uses Δ9-THC-analogs.”

Preparation of a Magnetic Particle/Recognition Molecule

In one embodiment of the invention, one or more magnetic particles are assembled near to, contiguous to, and/or bound to one or more recognition molecules. This construct may be prepared by means well known to those skilled in the art.

One such attachment process is described in U.S. Pat. No. 5,932,097, and also in European patent specification EP 0919285; the entire disclosure of each of these United States patent publications is hereby incorporated by reference into this specification.

Referring to U.S. Pat. No. 5,932,097, and at columns 16-19 thereof, reference is made to the “ATTACHMENT AND USE OF AFFINITY RECOGNITION MOLECULES BOUND TO MAGNETIC PARTICLES.” This section of U.S. Pat. No. 5,932,097, in relevant parts, is set forth below.

“As used herein, the term ‘affinity recognition molecule’ refers to a molecule that recognizes and binds another molecule by specific three-dimensional interactions that yield an affinity and specificity of binding comparable to the binding of an antibody with its corresponding antigen or an enzyme with its substrate. Typically, the binding is noncovalent, but the binding can also be covalent or become covalent during the course of the interaction. The noncovalent binding typically occurs by means of hydrophobic interactions, hydrogen bonds, or ionic bonds. The combination of the affinity recognition molecule and the molecule to which it binds is referred to generically as a “specific binding pair.” Either member of the specific binding pair can be designated the affinity recognition molecule; the designation is for convenience according to the use made of the interaction. One or both members of the specific binding pair can be part of a larger structure such as a virion, an intact cell, a cell membrane, or a subcellular organelle such as a mitochondrion or a chloroplast.” (See from line 66 of column 16 to line 17 of column 17).

“Examples of affinity recognition molecules in biology include antibodies, enzymes, specific binding proteins, nucleic acid molecules, and receptors. Examples of receptors include viral receptors and hormone receptors. Examples of specific binding pairs include antibody-antigen, antibodyhapten, nucleic acid molecule-complementary nucleic acid molecule, receptor-hormone, lectin-carbohydrate moiety, enzyme substrate, enzyme-inhibitor, biotin-avidin, and viruscellular receptor. One particularly important class of antigens is the Cluster of Differentiation (CD) antigens found on cells of hematopoietic origin, particularly on leukocytes, as well as on other cells. These antigens are significant in the activity and regulation of the immune system. One particularly significant CD antigen is CD34, found on stem cells. These are totipotent cells that can regenerate all of the cells of hematopoietic origin, including leukocytes, erythrocytes, and platelets.“ (See lines 18-34 of column 17.)

“As used herein, the term “antibody” includes both intact antibody molecules of the appropriate specificity and antibody fragments (including Fab, F(ab′), Fv, and F(ab′)2 fragments), as well as chemically modified intact antibody molecules and antibody fragments such as Fv fragments, including hybrid antibodies assembled by in vitro re-association of subunits. The term also encompasses both polyclonal and monoclonal antibodies. Also included are genetically engineered antibody molecules such as single chain antibody molecules, generally referred to as sFv. The term “antibody” also includes modified antibodies or antibodies conjugated to labels or other molecules that do not block or alter the binding capacity of the antibody.” (See lines 35-47 of column 17.)

“Methods for the covalent attachment of biological recognition molecules to solid phase surfaces, including the magnetic particles of the present invention, are well known in the art and can be chosen according to the functional groups available on the biological recognition molecule and the solid phase surface.” (See from line 64 of column 17 to line 2 of column 18.)

“Although, typically, the biological recognition molecules are covalently attached to the magnetic particles, alternatively, noncovalent attachment can be used. Methods for noncovalent attachment of biological recognition molecules to magnetic particles are well known in the art and need not be described further here.” (See lines 1-6 of column 19).

“Conjugation of biological recognition molecules to magnetic particles is described in U.S. Pat. No. 4,935,147 to Ullman et al., and in U.S. Pat. No. 5,145,784 to Cox et al., both of which are incorporated herein by this reference.” (See lines 7-10 of column 19 of U.S. Pat. No. 5,932,097.)

Bt way of further illustration, another means for constructing an assembly in which one or more recognition particles may be disposed on or near one or more magnetic particles is disclosed in both published United States patent application US 2003/0092069 and its corresponding European patent application 1262555; the entire disclosure of each of these patent documents is hereby incorporated by reference into this specification.

Referring to published United States patent application US 2003/0092069, such patent application claims:
“1. A hollow nano particle, comprising a protein capable of forming a particle, and a biorecognition molecule introduced thereto.”
“2. A hollow nano particle, comprising a protein particle obtained by expressing a protein in a eucaryotic cell, and a biorecognition molecule introduced thereto.”
“3. The hollow nano particle of claim 2, wherein the eucaryotic cell is either yeast or recombinant yeast.”
“4. The hollow nano particle of claim 2, wherein the eucaryotic cell is an insect cell.”
“5. The hollow nano particle of any one of claims 1 to 4, wherein the protein capable of forming a particle is a hepatitis B virus surface antigen protein.”
“6. The hollow nano particle of claim 5, wherein the hepatitis B virus surface antigen protein is one of which its antigenicity has been reduced.”
“7. The hollow nano particle of any one of claims 1 to 6, wherein the biorecognition molecule is a cell function-regulating molecule.”
“8. The hollow nano particle of any one of claims 1 to 6, wherein the biorecognition molecule is an antigen.”
“9. The hollow nano particle of any one of claims 1 to 6, wherein the biorecognition molecule is an antibody.”
“10. The hollow nano particle of any one of claims 1 to 6, wherein the biorecognition molecule is a sugar chain.”
“11. A transporter of substances, comprising the hollow nano particle of any one of claims 1 to 10, and a substance that is to be introduced into cells incorporated therein.”
“12. The transporter of substances of claim 11, wherein the substance is a gene.”
“13. The transporter of substances of claim 11, wherein the substance is a protein.”
“14. The transporter of substances of claim 11, wherein the substance is an RNase that shows cytotoxicity in the cell.”
“15. The transporter of substances of claim 11, wherein the substance is a compound.”
“16. A method of preparing the transporter of substances of any one of claims 11 to 15, comprising the insertion of the substance to the hollow nano particle of any one of claims 1 to 10 by electroporation.”
“17. A method for preparing the transporter of substances of any one of claims 11 to 15, comprising the insertion of the substance to the hollow nano particle of any one of claims 1 to 10 by ultrasonicaiton.”
“18. A method for preparing the transporter of substances of any of claims 11 to 15, comprising the insertion of the substance to the hollow nano particle of any one of claims 1 to 10 by simple diffusion.”
“19. A method for preparing the transporter of substances of any one of claims 11 to 15, comprising the insertion of the substance to the hollow nano particle of any one of claims 1 to 10 by using a charged lipid.”
“20. A method for transferring a substance into cells or tissues, which comprises the use of the hollow nano particles of any one of claims 1 to 10.”
“21. A method for transferring a substance into cells or tissues, which comprises the use of the transporter of substances of any one of claims 11 to 15.”
“22. A therapeutic method for treating diseases, which comprises transporting a substance to certain cells or tissues by using at least one method of transferring a substance of claims 20 or 21.”

Published United States patent application 2003/0092069 describes some of the recognition molecules (“biorecognition molecules”) that may preferably be used in the process of such patent application, stating that: “As the biorecognition molecules that is introduced into a protein capable of forming particles, for example, cell function-regulating molecules, that is, molecules that regulates cell function such as growth factor, cytokines, etc.; cell or tissue-recognizing molecules such as cell surface antigen, tissue specific antigen, receptor, etc.; molecules derived from viruses or microorganisms; antibodies, sugar chains, lipids, and the like may preferably be used. These maybe chosen properly according to the target cells or tissues.” (See page 2 of the published patent application.)

“According to the present invention, a substance desired to be introduced into target cells or tissues (substance to be introduced into cells) is incorporated in the protein hollow nano particles as described above, to form a transporter of a substance that shows cell specificity. The substance to be introduced into cells, which is incorporated in the transporter, includes, for example, genes such as DNAs and RNAs, natural or synthetic proteins, oligonucleotides, peptides, drugs, natural or synthetic compounds, and the like. Specifically, human RNase I (Jinno H., Ueda M., Ozawa S., Ikeda T., Enomoto K., Psarras K., Kitajima M., Yamada H., Seno M., Life Sci. 1996, 58 (21), 1901-8), and RNase 3 (also known as ECP: Eosinophil Cationic Protein; Mallorqui-Femandez G., Pous J., Peracaula R., Aymami J., Maeda T., Tada H., Yamada H., Seno M., de Llorens R., Gomis-Ruth F X, Coll M., J. Mol. Biol., Jul. 28, 2000, 300 (5), 1297-307), which has been reported by the present inventors are applicable.” (See the first two paragraphs of page 3 of the published patent application)

As is indicated in the next section of this specification, in one particular embodiment the “compound” delivered within the hollow nanoparticle (see claim 15 of US 2003/0092069) is a particular nanomagnetic material.

The Use of the Nanomagnetic Materials Described in WO 03/061755

In one preferred embodiment of the invention, one or more of the nanomagnetic materials described in International Publication Number WO 03/061755 is used in the constructs of this invention; the entire disclosure of such International Publication is hereby incorporated by reference into this specification.

In general, and as is known to those skilled in the art, nanomagnetic material is magnetic material which has an average particle size less than 100 nanometers and, preferably, in the range of from about 2 to 50 nanometers. Reference may be had, e.g., to U.S. Pat. No. 5,889,091 (rotationally free nanomagnetic material), U.S. Pat. Nos. 5,714,136, 5,667,924, and the like. The entire disclosure of each of these United States patents is hereby incorporated by reference into this specification.

As is disclosed in WO 03/061755 (see, e.g., page 8 thereof, the nanomagnetic materials may be, e.g., nano-sized ferrites such as, e.g., the nanomagnetic ferrites disclosed in U.S. Pat. No. 5,213,851, the entire disclosure of which is hereby incorporated by reference into this specification. As is disclosed on such page 8: “In general, and as is known to those skilled in the art, nanomagnetic material is magnetic material which has an average particle size less than 100 nanometers and, preferably, in the range of from about 2 to 50 nanometers. Reference may be had, e.g., to U.S. Pat. No. 5,889,091 (rotationally free nanomagnetic material), U.S. Pat. Nos. 5,714,136, 5,667,924, and the like. The entire disclosure of each of these United States patents is hereby incorporated by reference into this specification.

In one embodiment, the nanomagnetic material has one or more of the properties described at pages 10 et seq. of W0 03/061755. These and other pages of the publication are quoted in relevant part below.

“ . . . The layer of nanomagnetic particles 24 preferably has a saturation magnetization at 25 degrees Centigrade of from about 1 to about 36,000 Gauss, or higher. In one embodiment, the saturation magnetization at room temperature is from about 500 to about 10,000 Gauss.” (See page 10.)

“The nanomagnetic materials 24 typically comprise one or more of iron, cobalt, nickel, gadolinium, and sainarium atoms. Thus, e.g., typical nanomapetic materials include alloys of iron and nickel (pennalloy), cobalt, niobium, and zirconium (CNZ), iron, boron, and nitrogen, cobalt, iron, boron, and silica, iron, cobalt, boron, and fluoride, and the like. These and other materials are descried in a book by J. Douglas Adam, et al. entitled “Handbook of Thin Film Devices” (Academic Press, San Diego, Calif., 2000). Chapter 5 of this book beginning at page 185, describes “magnetic films for planar inductive components and devices;” and Tables 5.1 and 5.2 in this chapter describe many magnetic materials” (See the first full paragraph on page 11.)

“The nanomagnetic material 103 in film 104 also has a coercive force of from about 0.01 to about 5,000 Oersteds. The term coercive force refers to the magnetic field, H, which must be applied to a magnetic material in a symmetrical, cyclically magnetized fashion, to make the magnetic induction, B, vanish; this term often is referred to as magnetic coercive force. Reference may be had, e.g., to U.S. Pat. Nos. 4,061,824, 6,257,512, 5,967,522,35 4,939,610, 4,741,953, and the like. The entire disclosure of each of these United States patents is hereby incorporated by reference into this specification.” (See page 12.)

“ . . . The nanomagnetic material . . . preferably has a relative magnetic permeability of from about 1 to about 500,000; in one embodiment, such material . . . has a relative magnetic permeability of from about 1.5 to about 260,000 . . . . In one embodiment, the nanomagnetic material has a relative magnetic permeability of from about 1.5 to about 2,000.” (See page 13.)

“The nanomagnetic material 103 in film 104 preferably has a mass density of at least about 0.001 grams per cubic centimeter; in one embodiment, such mass density is at least about I gram per cubic centimeter. As used in this specification, the term mass density refers to the mass of a given substance eper unit volume. See, e.g., page 510 of the aforementioned “McGraw-Hill Dictionary of Scientific and Technical Ten-ns.” In one embodiment, the film 104 has a mass density of at least about 3 grams per cubic centimeter.” (See page 13.)

Nanomagnetic Materials Comprised of Aluminum and Iron Atoms

In one embodiment, the nanomagnetic material used contains both aluminum and iron atoms. These materials are described, e.g., on pages 43 et seq. of WO 03/061755. Selected portions of these pages are presented below.

“In one preferred embodiment of the invention, a sputtering technique is used to prepare an AlFe thin film as well as comparable thin films containing other atomic moieties, such as, e.g., elemental nitrogen, and elemental oxygen. Conventional sputtering techniques may be used to prepare such films by sputtering.. See, for example, R. Herrmann and G. Brauer, D. C.—and R. F. Magnetron Sputtering,” in the “Handbook of Optical Properties. Volume I—Thin Films for Optical Coatings,” edited by R. E. Hummel and K. H. Guenther (CRC Press, Boca Raton, Fla., 1955). Reference also may be had, e.g., to M. Allendorf, “Report of Coatings on Glass Technology Roadinap Workshop,” Jan. 18-19, 2000, Livermore, Calif.; and also to U.S. Pat. No. 6,342,134, “Method for producing piezoelectric films with rotating magnetron sputtering system.” The entire disclosure of each of these prior art documents is hereby incorporated by reference into this specification.” (See page 43.)

“The aforementioned process . . . may be adapted to produce other, comparable thin films, as is illustrated in FIG. 37. Referring to FIG. 37 . . . , a phase diagram 5000 is presented. As is illustrated by this phase diagram 5000, the nanomagnetic material used in the composition of this invention preferably is comprised of one or more moieties A, B, and C.” (See page 46.)

“The moiety A depicted in phase diagram 5000 is comprised of a magnetic element selected from the group consisting of a transition series metal, a rare earth series metal, or actinide metal, a mixture thereof, and/or an alloy thereof.” (See page 46.)

“The transition series metals include chromium, manganese, iron, cobalt, nickel. One may use alloys of iron, cobalt, and nickel such as, e.g., iron-aluminum, iron-carbon, iron-chromium, iron-cobalt, iron-nickel, iron nitride . . . , iron phosphide, iron-silicon, iron-vanadium, nickel-cobalt, nickel-copper, and the like. One may use alloys of manganese such as, e.g, manganese-aluminum, manganese-bismuth, MnAs, MnSb, MnTe, manganese-copper, manganese-gold, manganese-nickel, manganese-sulfur and related compounds, manganese-antimony, manganese-tin, manganese-zinc, Heusler alloy, and the like. One may use compounds and alloys of the iron group, including oxides of the iron group, halides of the iron group, boride of the transition elements, sulfides of the iron group, platinum and palladium with the iron group, chromium compounds, and the like.” (See page 46.)

“One may use a rare earth and/or actinide metal such as, e.g., Ce, Pr, Nd, Pm, Sm, Eu, Gd, Th, Dy, Ho, Er, Tm, Yb, Lu, La, mixtures thereof, and alloys thereof. One may also use one or ore of the actinides such as, e.g., Th, Pa, U, Np, Pu, Am, Cm, Bk, Cf, Es, Fm, Md, No, Lr, Ac, and the like.” (See page 47.)

“These moieties, compounds thereof, and alloys thereof are well known and are described, e.g., in the aforementioned text of R. S. Tebble et al. entitled “Magnetic Materials.” In one preferred embodiment, moiety A is selected from the group consisting of iron, nickel, cobalt, alloys thereof, and mixtures thereof In this embodiment, the moiety A is magnetic, i.e., it has a relative magnetic permeability of from about 1 to about 500,000. As is known to those skilled in the art, relative magnetic permeability is a factor, characteristic of a material, which is proportional to the magnetic induction produced in a material divided by the magnetic field strength; it is a tensor when these quantities are not parallel. See, e.g., page 4128 of E. U. Condon et al.'s “Handbook of Physics” (McGraw-Hill Book Company, Inc., New York, N.Y., 1958).” (See page 47.)

“The moiety A also preferably has a saturation magnetization of from about I to about 36,000 Gauss, and a coercive force of from about 0.01 to about 5,000 Oersteds.” See page 47.)

“The moiety A may be present in the nanomagnetic material either in its elemental form, as an alloy, in a solid solution, or as a compound.” (See page 47.)

“It is preferred at least about I mole percent of moiety A be present in the nanomagnetic material (by total moles of A, B, and C), and it is more preferred that at least IO mole percent of such moiety A be present in the nanomagnetic material (by total moles of A, B, and Q. In one embodiment, at least 60 mole percent of such moiety A is present in the nanomagnetic material, (by total moles of A, B, and C.) In addition to moiety A, it is preferred to have moiety B be present in the nanomagnetic material. In this embodiment, moieties A and B are admixed with each other. The mixture may be a physical mixture, it may be a solid solution, it may be comprised of an alloy of the A/B moieties, etc. In one embodiment, the magnetic material A is dispersed within nonmagnetic material B. This embodiment is depicted schematically in FIG. 38.” (See page 47.)

“Referring to FIG. 38, and in the preferred embodiment depicted therein, it will be seen that A moieties 5002, 5004, and 5006 are separated from each other either at the atomic level and/or at the nanometer level. The A moieties may be, e.g., A atoms, clusters of A atoms, A compounds, A solid solutions, etc; regardless of the form of the A moiety, it has the magnetic properties described hereinabove.” (See page 47.)

“In the embodiment depicted in FIG. 38, each A moiety produces an independent magnetic moment. The coherence length (L) between adjacent A moieties is, on average, from about 0.1 to about 100 nanometers and, more preferably, from about 1 to about 50 nanometers.” (See page 48.)

“Thus, referring again to FIG. 38, the normalized magnetic interaction between adjacent A moieties 5002 and 5004, and also between 5004 and 5006, is preferably described by the formula M=exp(−x/L), wherein M is the normalized magnetic interaction, exp is the base of the natural logarithm (and is approximately equal to 2.71828), x is the distance between adjacent A moieties, and L is the coherence length.

In one embodiment, and referring again to FIG. 38, x is preferably measured from the center 5001 of A moiety 5002 to the center 5002 of A moiety 5004; and x is preferably equal to from about 0.00001×L to about 100×L. In one embodiment, the ratio of x/L is at least 0.5 and, preferably, at least 1.5.” (See page 48.)

“Referring again to FIG. 37, the nanomagnetic material may be comprised of 100 percent of moiety A, provided that the such moiety A has the required normalized magnetic interaction (M). Alternatively, the nanomagnetic material may be comprised of both moiety A and moiety B. When moiety B is present in the nanomagnetic material, in whatever form or forms it is present, it is preferred that it be present at a mole ratio (by total moles of A and B) of from about I to about 99 percent and, preferably, from about 10 to about 90 percent. The B moiety, in whatever form it is present, is nonmagnetic, i.e., it has a relative magnetic permeability of 1.0; without wishing to be bound to any particular theory, applicants believe that the B moiety acts as buffer between adjacent A moieties. One may use, e.g., such elements as silicon, aluminum, boron, platinum, tantalum, palladium, yttrium, zirconium, titanium, calcium, beryllium, barium, silver, gold, indium, lead, tin, antimony, germanium, gallium, tungsten, bismuth, strontium, magnesium, zinc, and the like.” (See page 48.)

“In one embodiment, and without wishing to be bound to any particular theory, it is believed that B moiety provides plasticity to the nanomagnetic material that it would not have but for the presence of B. It is preferred that the bending radius of a substrate coated with both A and B moieties be at least 10 percent as great as the bending radius of a substrate coated with only the A moiety.” (See page 48.)

“The use of the B material allows one to produce a coated substrate with a springback angle of less than about 45 degrees. As is known to those skilled in the art, all materials have a finite modulus of elasticity; thus, plastic deforniationis followed by some elastic recovery when the load is removed. In bending, this recovery is called springback. See, e.g., page 462 of S. Kalparjian's “Manufacturing Engineering and Technology . . . .” (See pages 48 and 49.)

“Referring again to FIG. 37, and in one embodiment, the nanomagnetic material is comprised of moiety A, moiety C, and optionally moiety B. The moiety C is preferably selected from the group consisting of elemental oxygen, elemental nitrogen, elemental carbon, elemental fluorine, elemental chlorine, elemental hydrogen, and elemental helium, elemental neon, elemental argon, elemental krypton, elemental xenon, and the like.” (See page 49.)

“It is preferred, when the C moiety is present, that it be present in a concentration of from about I to about 90 mole percent, based upon the total number of moles of the A moiety and/or the B moiety and C moiety in the composition.” (See page 49.)

“Referring again to FIG. 37, and in the embodiment depicted, the area 5028 produces a composition which optimizes the degree to which magnetic flux are initially trapped and/or thereafter released by the composition when a magnetic field is withdrawing from the composition.” (See page 49.)

“Thus, one may optimize the A/B/C composition to preferably be within the area 5028. In general, the A/B/C composition has molar ratios such that the ratio of A/(A and C) is from about 1 to about 99 mole percent and, preferably, from about 10 to about 90 mole percent. In one preferred embodiment, such ratio is from about 40 to about 60 molar percent. The molar ratio of A/(A and B and C) generally is from about 1 to about 99 mole percent and, preferably, from about 10 to about 90 molar percent. In one embodiment, such molar ratio is from about 30 to about 60 molar percent. The molar ratio of B/(A plus B plus C) generally is from about 1 to about 99 mole percent and, preferably, from about 10 to about 40 mole percent, The molar ratio of C/(A plus B plus C) generally is from about 1 to about 99 mole percent and, preferably, from about 10 to about 50 mole percent.” (See page 50.)

Other Magnetic Materials that May be Used with the Recognition Molecule(s)

In addition to the nanomagnetic materials described in the aforementioned section on this specification, or instead of, one may use other magnetic materials.

Thus, e.g., one may use the superparamagnetic particles described in U.S. Pat. No. 4,770,183; the entire disclosure of this United States patent is hereby incorporated by reference into this specification. This United States patent claims: “An improved method for obtaining an in vivo NMR image or an organ or tissue of an animal or human subject, wherein the improvement comprises administering to such a subject as a contrast agent to enhance such NMR image an effective amount of a dispersoid which comprises uncoated, biodegradable superparamagnetic metal oxide particles dispersed in a physiologically acceptable carrier, an individual particle (i) comprising one or more biodegradable metal oxide crystals, each crystal about 10 to about 500 angstroms in diameter; (ii) having an overall means diameter of about 10 angstroms to about 5000 angstroms as measured on a Coulter particle size analyzer; and (iii) further characterized as having a retention time in said organ or tissue sufficiently long to permit an image to be obtained and being ultimately biodegraded in said organ or tissue within a period of about 7 days.”

At column 5 of U.S. Pat. No. 4,770,183, the following discussion of superparamagnetism occurs: “Superparamagnetic materials possess some properties characteristic of paramagnetic materials and some properties characteristic of ferromagnetic materials. Like paramagnetic particles, superparamagnetic particles rapidly lose their magnetic properties in the absence of an applied magnetic field; yet they also possess the high magnetic susceptibility found in ferromagnetic materials. Iron oxides such as magnetite or gamma ferric oxide exhibit superparamagnetism when the crystal diameter falls significantly below that of ferromagnetic materials. For cubic magnetite (Fe3 O4) this cut-off is a crystal diameter of about 300 angstroms [Dunlop, J. Geophys. Rev. 78 1780 (1972)]. A similar cut-off applies for gamma ferric oxide [Bate in Ferromagnetic Materials, vol. 2, Wohlfarth (ed.) (1980) p. 439]. Since iron oxide crystals are generally not of a single uniform size, the average size of purely ferromagnetic iron oxides is substantially larger than the cut-off of 300 angstroms (0.03 microns). For example, when gamma ferric oxide is used as a ferromagnetic material in magnetic recording, (Pfizer Corp. Pf 2228), particles are needle-like and about 0.35 microns long and 0.06 microns thick. Other ferromagnetic particles for data recording are between 0.1 and 10 microns in length [Jorgensen, The Complete Handbook of Magnetic Recording, p. 35 (1980)]. For a given type of crystal, preparations of purely ferromagnetic particles have average dimensions many times larger than preparations of superparamagnetic particles. The theoretical basis of superparamagnetism has been described in detail by Bean and Livingston [J. Applied Physics, Supplement to volume 30, 1205 (1959)]. Fundamental to the theory of superparamagnetic materials is the destabilizing effect of temperature on their magnetism. Thermal energy prevents the alignment of the magnetic moments present in superparamagnetic particles. After the removal of an applied magnetic field, the magnetic moments of superparamagnetic materials still exist but they are in rapid motion. Temperature also limits the magnetization of superparamagnetic materials produced by an applied magnetic field. At the temperatures of biological systems and in the applied magnetic fields of NMR imagers, superparamagnetic materials are less magnetic than their ferromagnetic counterparts. For example, Berkowitz, et al. (J. App. Phys. 39, 1261 (1968)] have noted decreased magnetism of small superparamagnetic iron oxides. This may in part explain why workers in the field of NMR imaging have looked to ferromagnetic materials as contrast agents on the theory that the more magnetic a material is per gram, the more effective that material should be in depressing T2 [Drain, Proc. Phys. Soc. 80, 1380 (1962); Dias and Lautebur, Mag. Res. Med. 3, 328 (1986)].”

By way of further illustration, one may use the colloidal, biodegradable, superparamagnetic contrast agent described in claim 21 of U.S. Pat. No. 5,679,323; the entire disclosure of this United States patent is hereby incorporated by reference into this specification. Such claim 21 describes: “21. A colloidal biodegradable superparamagnetic contrast agent, said contrast agent comprising (1) biodegradable superparamagnetic metal oxide particles, physically or chemically joined with (2) a ligand, wherein such metal oxide particles comprise one or more individual biodegradable superparamagnetic metal oxide crystals, and are capable of being biodegraded in such subject, as evidenced by a return of proton relaxation rates of the liver to pre-administration levels, within 30 days of administration; and wherein such ligand is capable of being recognized and internalized by hepatocytes of the liver by receptor mediated endocytosis, thereby making said metal oxide particles capable of being internalized by such hepatocytes, and is selected from the group consisting of (i) arabinogalactan, and (ii) a macromolecular species conjugate, which macromolecular species conjugate comprises two macromolecular species, a first macromolecular species which is arabinogalactan, and a second macromolecular species which is physically or chemically joined with the metal oxide particles and conjugated to the first macromolecular species.” The “ABSTRACT” of U.S. Pat. No. 5,679,323 more generally describes the inventions of the patent as including: “A new class of magnetic resonance (MR) contrast agents are described whose in vivo biodistribution is based upon the ability of certain cells to recognize and internalize macromolecules, including the MR contrast agents of the present invention, via a process which substantially involves receptor mediated endocytosis. The RME-type MR contrast agents described herein comprised of biodegradable superparamagnetic metal oxides associated with a variety of macromolecular species, including but not limited to, serum proteins, hormones, asialoglycoproteins, galactose-terminal species, polysaccharides, arabinogalactan, or conjugates of these molecules with other polymeric substances such as a poly(organosilane) and dextran. One of the advantages of these MR contrast agents is that they may be selectively directed to those cells which bear receptors for a particular macromolecule or ligand and are capable of undergoing receptor mediated endocytosis. An MR contrast agent prepared from biodegradable superparamagnetic iron oxide and asialofetuin, or more preferably arabinogalactan, for example, is selectively localized in the hepatocytes the liver with no significant accumulation in the spleen. An MR experiment which can be carried out shortly after administration to the subject of the contrast agents of the invention can thus provide a method for obtaining an enhanced MR image, as well as valuable information regarding the functional or metabolic state of the organ or tissue under examination. Preparative methods, biodistribution data, and time function MR images are further provided.”

By way of further illustration, one may use the iron-containing nanoparticles disclosed in U.S. Pat. No. 6,048,515 with the recognition molecule(s); the entire disclosure of this United States patent is hereby incorporated by reference into this specification. As is disclosed in the “ABSTRACT” of U.S. Pat. No. 6,048,515, “Modular iron-containing nanoparticles are disclosed, as well as their production and use in diagnosis and therapy. The nanoparticles are characterized in that they have an iron-containing core, a primary coat (synthetic polymer) and a secondary coat (target polymer), and optional auxiliary pharmaceutical substances, pharmaceuticals and/or adsorption mediators.”

By way of further illustration, one my use magnetic particles having two antiparallel ferromagnetic layers that are disclosed in U.S. Pat. No. 6,337,215, together with one or more of the recognition molecules (“affinity recognition molecules.” This United States patent describes and claims: “1. A composition of matter comprising: a magnetic particle comprising a first ferromagnetic layer having a moment oriented in a first direction, a second ferromagnetic layer having a moment oriented in a second direction generally antiparallel to said first direction, and a nonmagnetic spacer layer located between and in contact with the first and second ferromagnetic layers, and wherein the magnitude of the moment of the first ferromagnetic layer is substantially equal to the magnitude of the moment of the second ferromagnetic layer so that the magnetic particle has substantially zero net magnetic moment in the absence of an applied magnetic field, and wherein the thickness of the magnetic particle is substantially the same as the total thickness of said layers making up the particle; a coating on the surface of the magnetic particle; and an affinity recognition molecule attached to the coating of the magnetic particle for selectively binding with a target molecule.”

At columns 16 et seq. of U.S. Pat. No. 6,337,215, means are disclosed for binding “affinity recognition molecules” to magnetic particles. Relevant portions from these columns of the patent are set forth below.

“The following sections discuss the use of the above identified magnetic particles as nuclei for affinity molecules that are bound to the magnetic particles of the present invention. As indicated above, magnetic particles according to the present invention are attached to at least one affinity recognition molecule. As used herein, the term “affinity recognition molecule” refers to a molecule that recognizes and binds another molecule by specific three-dimensional interactions that yield an affinity and specificity of binding comparable to the binding of an antibody with its corresponding antigen or an enzyme with its substrate. Typically, the binding is noncovalent, but the binding can also be covalent or become covalent during the course of the interaction. The non-covalent binding typically occurs by means of hydrophobic interactions, hydrogen bonds, or ionic bonds. The combination of the affinity recognition molecule and the molecule to which it binds is referred to generically as a “specific binding pair.” Either member of the specific binding pair can be designated the affinity recognition molecule; the designation is for convenience according to the use made of the interaction. One or both members of the specific binding pair can be part of a larger structure such as a virion, an intact cell, a cell membrane, or a subcellular organelle such as a mitochondrion or a chloroplast.”

“Examples of affinity recognition molecules in biology include antibodies, enzymes, specific binding proteins, nucleic acid molecules, and receptors. Examples of receptors include viral receptors and hormone receptors. Examples of specific binding pairs include antibody-antigen, antibodyhapten, nucleic acid molecule-complementary nucleic acid molecule, receptor-hormone, lectin-carbohydrate moiety, enzyme substrate, enzyme-inhibitor, biotin-avidin, and viruscellular receptor. One particularly important class of antigens is the Cluster of Differentiation (CD) antigens found on cells of hematopoietic origin, particularly on leukocytes, as well as on other cells. These antigens are significant in the activity and regulation of the immune system. One particularly significant CD antigen is CD34, found on stem cells. These are totipotent cells that can regenerate all of the cells of hematopoietic origin, including leukocytes, erythrocytes, and platelets.”

“As used herein, the term “antibody” includes both intact antibody molecules of the appropriate specificity and antibody fragments (including Fab, F(ab′), Fv, and F(ab′)2 fragments), as well as chemically modified intact antibody molecules and antibody fragments such as Fv fragments, including hybrid antibodies assembled by in vitro reassociation of subunits. The term also encompasses both polyclonal and monoclonal antibodies. Also included are genetically engineered antibody molecules such as single chain antibody molecules, generally referred to as sFv. The term “antibody” also includes modified antibodies or antibodies conjugated to labels or other molecules that do not block or alter the binding capacity of the antibody.”

“Methods for the covalent attachment of biological recognition molecules to solid phase surfaces, including the magnetic particles of-the present invention, are well known in the art and can be chosen according to the functional groups available on the biological recognition molecule and the solid phase surface.”

“Many reactive groups on both protein and non-protein compounds are available for conjugation. For example, organic moieties containing carboxyl groups or that can be carboxylated can be conjugated to proteins via the mixed anhydride method, the carbodiimide method, using dicyclohexylcarbodiimide, and the N hydroxysuccinimide ester method. If the organic moiety contains amino groups or reducible nitro groups or can be substituted with such groups, conjugation can be achieved by one of several techniques. Aromatic amines can be converted to diazonium salts by the slow addition of nitrous acid and then reacted with proteins at a pH of about 9. If the organic moiety contains aliphatic amines, such groups can be conjugated to proteins by various methods, including carbodiimide, tolylene-2,4-diisocyanate, or malemide compounds, particularly the N-hydroxysuccinimide esters of malemide derivatives. An example of such a compound is 4 (Nmaleimidomethyl)-cyclohexane-1-carboxylic acid. Another example is m-male imidobenzoyl-N-hydroxysuccinimide ester. Still another reagent that can be used is N-succinimidyl-3 (2-pyridyldithio) propionate. Also, bifunctional esters, such as dimethylpimelimidate, dimethyladipimidate, or dimethylsuberimidate, can be used to couple amino-group containing moieties to proteins.”

“Additionally, aliphatic amines can also be converted to aromatic amines by reaction with p-nitrobenzoylchloride and subsequent reduction to a p-aminobenzoylamide, which can then be coupled to proteins after diazotization.”

“Organic moieties containing hydroxyl groups can be cross-linked by a number of indirect procedures. For example, the conversion of an alcohol moiety to the half ester of succinic acid (hemisuccinate) introduces a carboxyl group available for conjugation. The bifunctional reagent sebacoyldichloride converts alcohol to acid chloride which, at pH 8.5, reacts readily with proteins. Hydroxyl containing organic moieties can also be conjugated through the highly reactive chlorocarbonates, prepared with an equal molar amount of phosgene.”

“For organic moieties containing ketones or aldehydes, such carbonyl-containing groups can be derivatized into carboxyl groups through the formation of O-(carboxymethyl) oximes. Ketone groups can also be derivatized with p-hydrazinobenzoic acid to produce carboxyl groups that can be conjugated to the specific binding partner as described above. Organic moieties containing aldehyde groups can be directly conjugated through the formation of Schiff bases which are then stabilized by a reduction with sodium borohydride.”

“One particularly useful cross-linking agent for hydroxyl-containing organic moieties is a photosensitive noncleavable heterobifunctional cross-linking reagent, sulfosuccinimidyl 6-[4¢-azido-2¢-nitrophenylanmino]hexanoate. Other similar reagents are described in S. S. Wong, “Chemistry of Protein Conjugation and CrossLinking,” (CRC Press, Inc., Boca Raton, Fla. 1993). Other methods of crosslinking are also described in P. Tijssen, “Practice and Theory of Enzyme Immunoassays” (Elsevier, Amsterdam, 1985), pp. 221-295.”

“Other cross-linking reagents can be used that introduce spacers between the organic moiety and the biological recognition molecule. The length of the spacer can be chosen to preserve or enhance reactivity between the members of the specific binding pair, or, conversely, to limit the reactivity, as may be desired to enhance specificity and inhibit the existence of cross-reactivity.”

“Although, typically, the biological recognition molecules are covalently attached to the magnetic particles, alternatively, non-covalent attachment can be used. Methods for non-covalent attachment of biological recognition molecules to magnetic particles are well known in the art and need not be described further here.”

“Conjugation of biological recognition molecules to magnetic particles is described in U.S. Pat. No. 4,935,147 to Ullman et al., and in U.S. Pat. No. 5,145,784 to Cox et al., both of which are incorporated herein by this reference.”

By way of yet further illustration, one may use the ultrafine, lightly coated superparamagnetic particles disclosed in U.S. Pat. No. 6,207,134 together with one or more recognition molecules; the entire disclosure of U.S. Pat. No. 6,207,134 is hereby incorporated by reference into this specification.

U.S. Pat. No. 6,207,134 describes and claims: “A diagnostic agent comprising a composite particulate material the particles whereof comprise a diagnostically effective, substantially water-insoluble, metal oxide crystalline material and a polyionic coating agent, wherein said particles have a size of below 300 nm, said crystalline material has a crystal size of from 1 to 100 nm, the weight ratio of said crystalline material to said coating agent is in the range 1000:1 to 11:1, and said coating agent is selected from the group consisting of natural and synthetic structural-type polysaccharides, synthetic polyaminoacids, physiologically tolerable synthetic polymers and derivatives thereof.” As is more generally described in the “ABSTRACT” of such patent, “The invention relates to particulate contrast agents, especially contrast agents for MR imaging having a metal oxide core which is preferably superparamagnetic iron oxide. The particulate contrast agents are provided with a low coating density of a polyelectrolyte coating agent selected from structural polysaccharides and synthetic polymers, especially polyaminoacids. Unlike conventional coated particulates, the new particles have reduced or no effect on cardiovascular parameters, platelet depletion, complement activation and blood coagulation.”

By way of yet further illustration, one may use the heat stable colloidal iron oxides disclosed in U.S. Pat. No. 6,599,498 together with one or more recognition molecules; the entire disclosure of this United States patent is hereby incorporated by reference into this specification. As is indicated in the “ABSTRACT” of this patent, the contrast agents of this patent comprise “ . . . carboxyalkylated reduced polysaccharides coated ultrasmall superparamagnetic iron oxides.”

The Use of Microbubble Shell Delivery Systems

In one embodiment, the recognition molecule(s) and/or the magnetic particles are delivered by a microbubble-shell binding moiety, such as that disclosed in U.S. Pat. No. 6,245,318. The entire disclosure of such U.S. patent is hereby incorporated by reference into this specification.

U.S. Pat. No. 6,245,318 describes and claims: “. An ultrasound contrast media composition comprising a monolayer microbubble shell and a composition of the general formula: A-P-L, wherein A is an ultrasound contrast agent microbubble-shell binding moiety wherein anchor molecule A of the AP-L structure is anchored to the monolayer microbubble shell at the gas-liquid interface with said A-P-L structure intact during anchoring; P is a polymeric spacer arm having more than 10 monomer units; and L is a ligand, whereby said polymeric spacer arm provides spatial separation of the ligand from said microbubble.

As will be apparent, a ligand is a recognition molecule. Some of these ligands/recognition molecules are described at column 3 of U.S. Pat. No. 6,245,318. Thus, and as is disclosed in this column 3: “The ligand for use with the invention can be a biomolecule. Biomolecule refers to all natural and synthetic molecules that play a role in biological systems. Biomolecules include hormones, amino acids, vitamins, peptides, peptidomimetics, proteins, deoxyribonucleic acid (DNA) ribonucleic acid (RNA), lipids, albumins, polyclonal antibodies, receptor molecules, receptor binding molecules, monoclonal antibodies, carbohydrates and aptamers. Specific examples of biomolecules include insulins, prostaglandins, cytokines, chemokines, growth factors including angiogenesis factors, liposomes and nucleic acid probes. The advantages of using biomolecules include enhanced tissue targeting through specificity and delivery. Coupling of the chelating moieties to biomolecules can be accomplished by several known methods (e.g., Krejcarek and Tucker Biochem. Biophys. Res. Comm, 30, 581 (1977); Hnatowich, et al. Science, 220, 613 (1983). For example, a reactive moiety present in one of the R groups is coupled with a second reactive group located on the biomolecule. Typically, a nucleophilic group is reacted with an electrophilic group to form a covalent bond between the biomolecule and the chelate. Examples of nucleophilic groups include amines, anilines, alcohols, phenols, thiols and hydrazines. Electrophilic group examples include halides, disulfides, epoxides, maleimides, acid chlorides, anhydrides, mixed anhydrides, activated esters, imidates, isocyanates and isothiocyanates. Biomolecules can be covalently or noncovalently attached to one of the tips of the polymer chain, while the lipid anchor grouping is attached to the other end of this polymer chain.”

“A specific example of using the claimed invention is tumor targeting. Ligands designed to bind specifically to receptors for angiogenesis factors expressed in tumor microvasculature and coupled to echogenic contrast agents enhance the specificity and sensitivity of ultrasound tumor detection. Angiogenesis is a process associated with tumor growth. Several peptides have been identified as promoters of angiogenesis including interleukins 8, and 6, acidic FGF, basic FGF, TNF-alpha, TGF-alpha, TGF-beta, and VEGF/VPF. See Rak, J. W.; St. Croix B. D. and Kerbel R. S. (1995), Anti-Cancer Drugs 6, p. 3-18. See also Bicknell, R. (1994), Annals of Oncology 5 (Suppl. 4), p. 545-550. Since angiogenesis is a process not generally carried out in the body except during wound healing and a few other specialized circumstances, ligands designed from angiogenesis factors will selectively target tumor vasculature with high specificity. A specific ligand useful for targeting tumor vasculature is the chemokine IL-8 or an analog, homolog, derivative or fragment thereof, or a peptide having specificity for a receptor of interluekin 8. Particularly useful are the amino acid residues at the N terminal end of IL-8, including the “ELR” sequence gluleu-arg found immediately before the initial cysteine residue. It is known that the ELR amino acid sequence of IL-8 is important for the binding interaction with its receptor. The ELR motif also apparently imparts the angiogenic properties of IL-8. See Strieter, R. M.; Kunkel, S. L.; Palverini, P. J.; Arenberg, D. A.; Waltz, A.; Opdenakker, G. and Van Damme, J. (1995), Journal of Leukocyte Biology 576, p. 752-762. For the complete sequence of IL-8 see U.S. Pat. No. 5,436,686 Sep. 13, 1994, incorporated herein by reference.”

Referring again to the disclosure of U.S. Pat. No. 6,245,318, the monolayer microbubble-shells of this patent are described at columns 3-4. Referring to these columns, it will be seen that: “Monolayer microbubble-shells include any composition suitable for ultrasound imaging and capable of being gas filled, liquid filled, or combinations of gas and liquid, and includes those with a protein shell, natural polymer shell, synthetic polymer shell, surfactant, lipid, phospholipid, sphingolipid, sulfolipid, oligolipid, polymeric lipid, sterol, terpene, fullerene, wax, or hydrocarbon shell or any combination of these.

Gases, liquids, and combinations thereof suitable for use with the invention include decafluorobutane, dctafluorocyclobutane, decafluoroisobutane, octafluoropropane, octafluorocyclopropane, dodecafluoropentane, decafluorocyclopentane, decafluoroisopentane, perfluoropexane, perfluorocyclohexane, perfluoroisohexane, sulfur hexafluoride, and perfluorooctaines, perfluorononanes; perfluorodecanes, optionally brominated.”

“Generally, in making microbubbles, an aqueous dispersion of phospholipid (DSPC), surfactant (PEG stearate) and biotinamidocaproyl PEG-DSPE are mixed in an organic solvent, then the solvent evaporated and saline added. After that, the mixture is generally blended (e.g. sonication, colloid mill) in order to create an aqueous dispersion of the components, and then blending is continued in the presence of the flow of a gas such as decafluorobutane gas, which is dispersed in the form of microbubbles in the aqueous phase. At that moment, DSPC, PEGstearate and Bac-PEG-DSPE are deposited on the gasaqueous interface, with hydrophobic residue facing the gas phase and hydrophilic part of the molecule (including the ligand part) immersed in the aqueous phase. In such a way, the whole surface of the microbubble formed is covered by these molecules which thus create a protective shell.”

In one embodiment of the instant invention, the interior of the microbubble shell is filled with a gaseous and/or a liquid magnetic material, and the exterior of the microbubble shell is bonded directly or indirectly to the recognition molecule (the ligand).

Use of a Conjugated Fullerene as a Delivery System

In one embodiment of this invention, a fullerene conjugated to a recognition molecule is used, with our without magnetic material, as a delivery system.

U.S. Pat. No. 5,688,486 contains at least the following claims:

1. A compound comprising a curved or planar molecular mesh structure in the skeleton of which essentially all mesh aperture ring atoms are branching sites, said compound being externally linked to a metal or metal complex coordinating chelant group, or a conjugate, intercalate, inclusion compound or salt thereof, for use as a diagnostic or therapeutic agent.

4. A compound as claimed in claim 1 comprising a fullerene or fullerene derivative.

5. A compound as claimed in claim 4 comprising a Mn @ Cm fullerene derivative (wherein n and m are positive integers and M is a metal) or a conjugate or salt thereof.

6. A compound as claimed in claim 5 wherein m is an even number 44 to 112, and n is 1, 2, 3 or 4.

7. A compounds as claimed in claim 6 wherein m is 60, 70, 80 or 82 and n is 1 or 2.

8. A compound as claimed in claim 5 wherein at least one M in Mn @ Cm is paramagnetic.

9. A compound as claimed in claim 2 comprising a @ Cm Haln' fullerene halide (wherein m and n′ are positive integers and Hal is F, Cl, Br or I) or a conjugate, inclusion compound or salt thereof.

10. A compound comprising a curved or planar molecular mesh structure in the skeleton of which essentially all mesh aperture ring atoms are branching sites, said compound being externally linked to a metal or metal complex coordinating chelant group, or a conjugate, intercalate, inclusion compound or salt thereof, for use as a diagnostic or therapeutic agent, comprising a metallo-carbohedrane or a derivative thereof.

11. A compound as claimed in claim 10 comprising a @ M'm.sbsb.1 Cm.sbsb.2metallo-carbohedrane (wherein M′ is a transition metal and m1 and m2 are positive integers) or a derivative thereof.

12. A compound comprising a curved or planar molecular mesh structure in the skeleton of which essentially all mesh aperture ring atoms are branching sites, said compound being externally linked to a metal or metal complex coordinating chelant group, or a conjugate, intercalate, inclusion compound or salt thereof, for use as a diagnostic or therapeutic agent, comprising a Gd encapsulating molecular mesh structure.

14. A compound comprising a curved or planar molecular mesh structure in the skeleton of which essentially all mesh aperture ring atoms are branching sites, said compound being externally linked to a metal or metal complex coordinating chelant group, or a conjugate, intercalate, inclusion compound or salt thereof, for use as a diagnostic or therapeutic agent, comprising at least one curved molecular mesh structure linked to a metal or metal complex coordinating chelant group.

15. A compound as claimed in claim 14 comprising at least one metal complex coordinating chelant group wherein the chelated complex comprises at least two metal atoms.

16. A compound as claimed in claim 12 being Gdn @ Cm (where n and m are positive integers) optionally water-solubilized by derivatisation or carrier enclosure.

17. A compound comprising a curved or planar molecular mesh structure in the skeleton of which essentially all mesh aperture ring atoms are branching sites, said compound being externally linked to a metal or metal complex coordinating chelant group, or a conjugate, intercalate, inclusion compound or salt thereof, for use as a diagnostic imaging contrast agent.

18. A diagnostic composition comprising a sterile pharmaceutically acceptable carrier or excipient together with an image contrast enhancing physiologically tolerable compound comprising a curved or planar molecular mesh structure in the skeleton of which essentially all mesh aperture ring atoms are branching sites, said compound being externally linked to a metal or metal complex coordinating chelant group, or a conjugate, intercalate, inclusion compound or salt thereof.

19. A composition as claimed in claim 18 wherein said compound contains a diagnostically effective moiety.

20. A composition as claimed in claim 19 wherein said moiety is enclosed by said molecular mesh structure.

21. A composition as claimed in claim 19 wherein said moiety is attached to said molecular mesh structure.

22. A composition as claimed in claim 19 wherein said moiety forms part of the skeleton of said molecular mesh structure.

23. A composition as claimed in claim 19 wherein said moiety is selected from radiolabels, magnetic labels, elements of atomic number greater than 50, chromophores and fluorophores.

25. A pharmaceutical composition comprising a sterile pharmaceutically acceptable carrier or excipient together with a therapeutically effective compound comprising a curved molecular mesh structure in the skeleton of which essentially all mesh aperture ring atoms are branching sites, said compound being externally linked to a metal or metal complex coordinating chelant group, or a conjugate, intercalate, inclusion compound or salt thereof.

26. A composition comprising a sterile pharmaceutically acceptable carrier or excipient together with a therapeutically effective compound comprising a curved molecular mesh structure in the skeleton of which essentially all mesh aperture ring atoms are branching sites, said compound being externally linked to a metal or metal complex coordinating chelant group, or a conjugate, intercalate, inclusion compound or salt thereof wherein said compound contains a therapeutically effective metal.

27. A composition as claimed in claim 25 wherein said compound contains a therapeutically active entity releasably conjugated to said molecular mesh structure.

28. A composition as claimed in claim 25 wherein said compound comprises a photo-activatable therapeutic entity.”

The fullerene structures of U.S. Pat. No. 5,688,486 can be conjugated to one or more recognition molecules. Thus, as is disclosed in this patent, “Where a diagnostic or therapeutic entity is to be carried by the mesh structure, this may be achieved in at least four ways: skeleton atoms in the mesh structure (e.g. carbon atoms in a carbon allotrope) may be derivatised to bind the diagnostic or therapeutic entity directly or indirectly to the skeleton; diagnostically or therapeutically effective atoms may be substituted for framework atoms (as for example in the boron-doped fullerenes and the met-cars); the diagnostic or therapeutic entity may be intercalated between adjacent webs (as for example in graphite, a buckytube or an amorphous carbon); or the diagnostic or therapeutic entity may be entrapped within a cage-like mesh (as for example within a buckyball). Moreover in each case the skeleton may be derivatised to enhance other properties of the macromolecule, e.g. to include hydrophilic or lipophilic groups or biologically targeting groups or structures. Examples of macromolecules, biomolecules and macrostructures to which the mesh structure may be conjugated in this regard include polymers (such as polylysine or polyethyleneglycol), dendrimers (such as 1st to 6th generation starburst dendrimers, in particular PAMAM dendrimers), polysaccharides, proteins, antibodies or fragments thereof (especially monoclonal antibodies and fragments such as Fab fragments thereof), glycoproteins, proteoglycans, liposomes, aerogels, peptides, hormones, steroids, microorganisms, human or non-human cells or cell fragments, cell adhesion molecules (in particular nerve adhesion molecules such as are described in WO-A-92/04916), other biomolecules, etc.) to assist in the achievement of a desired biodistribution. Generally, such derivatization will most conveniently be achieved by introduction of amine or hydroxyl functions to which the macromolecule, biomolecule, etc can be bound either directly or via a linker molecule, e.g. a bi or polyfunctional acid, activated acid or oxirane.”

In one embodiment, both a recognition molecule and magnetic material is bound to the fullerene structure.

A Preferred Class of Nanomagnetic Particles

In one embodiment of this invention, a class of nanomagnetic particles with certain properties is used. These particles generally have a particle size distribution such that at least about 50 percent of such particles have an average crystallite size of from about 3 to about 10 nanometers, as measured by X-ray diffraction and transmission electron microscopy. In one embodiment, at least 60 percent of such particles have an average crystallite size of from about 4 to about 10 nanometers. In another embodiment, at least about 80 percent of such particles have an average crystallite size of from 6 to about 10 nanometers. In yet another embodiment, the average crystallite size is from about 7 to about 10 nanometers.

The coercive magnetic force, Hc, of the nanomagnetic particles preferably ranges from about 1 to about 200 Oersteds. This coercive magnetic force may be determined, e.g., by hysteresis loop analysis, measured by SQUID (superconducting quantum interference device) or VSM analyses. Reference may be had, e.g., to an article by Xingwu Wang et al on “NANO-MAGNETIC FeAl and FeAlN THIN FILMS VIA SPUTTERING,” presented at the 27th International Cocoa Beach Conference on Advanced Ceramics and Composites in Ceramic Engineering & Science Proceedings, Volume 24, Issue 3, 2003, at pages 629-636. Reference also may be had to an article by Xingwu Wang et al. on “Nano-Magnetic Coatings on Metallic Wires, given at the International Wire & Cable Symposium, Proceedings of the 52nd IWCS/Focus, Philadelphia, Pa., November, 2003, at pages 647-653.

Referring again to the coercive force, Hc, of the nanomagnetic particles, in one embodiment such coercive force is from about 10 to about 120 Oersteds and, more preferably, from about 20 to about 110 Oersteds.

The nanomagnetic particles preferably have a remnant magnetization (4×pixMr) of from about 10 to about 10,000 Gauss. In one embodiment, this remnant magnetization preferably is from about 1,000 to about 8,000 Gauss. In another embodiment, this remnant magnetization is from about 2,000 to about 7,5000 Gauss.

The nanomagnetic particles preferably have a saturation magnetization of from about 100 to about 24,000 Gauss. In one embodiment, the saturation magnetization is from about 10,000 to about 21,000 Gauss.

These magnetic properties, and the means for evaluating them, are well known to those skilled in the art. Reference may be had, e.g., to International Publication No. WO 03/061755, which is also referred to elsewhere in this specification. Reference also may be had, e.g., to U.S. Pat. No. 4,600,675, 4,946,374, 4,624,883, 4,617,234, 4,554,606, and 4,047,983 (all of which discuss the coercive force parameter), U.S. Pat. No. 6,344,955 (which discusses the remnant magnetization parameter), U.S. Pat. No. 4,705,613, 4,880,514, and 5,635,589 (all of which discuss the saturation magnetization parameter), and the like. The entire disclosure of each of these United States patents is hereby incorporated by reference into this specification.

The Use of Nanomagnetic Particles as Contrast Agents

Several small conducting rings were coated with a series of coatings, and a series of experiments was run as follows: In experiments 1-7, coatings comprised of nanomagnetic material were deposited onto copper rings; in control sample 8, the copper ring was uncoated. The samples are described with more particularity below.

Sample 1 was a three-layer coating of AlN—FeAlN—AlN. The first AlN was 400 nanometers thick. The FeAlN was 400 nanometers thick. The last AlN was 700 nanometers thick. The target material was 65 weight percent of Fe and 35 weight percent of Al.

Sample 2 was a three-layer coating of AlN—FeN—AlN. The first AlN was 400 nanometers thick. The FeN was 400 nanometers thick. The last AlN was 700 nanometers thick. The target material was 100% weight percent of Fe and 0 weight percent of Al.

Sample 3 was a three-layer coating of AlN—FeAlN—AlN. The first AlN was 400 nanometers thick. The FeAlN was 400 nanometers thick. The last AlN was 700 nanometers thick. The target material was 10 weight percent of Fe and 90 weight percent of Al.

Sample 4 was a two-layer coating of FeAlN—AlN. The FeAlN was 800 nanometers thick. The AlN was 700 nanometers thick. The target material was 10 weight percent of Fe and 90 weight percent of Al.

Sample 5 was a two coating of FeAlN—AlN. The FeAlN was 800 nanometers thick. The AlN was 700 nanometers thick. The target material was 82.5 weight percent of Fe and 17.5 weight percent of Al.

Sample 6 was a three-layer coating of AlN—FeAlN—AlN. The first AlN was 400 nanometers thick. The FeAlN was 400 nanometers thick. The last AlN was 700 nanometers thick. The target material was 82.5 weight percent of Fe and 17.5 weight percent of Al.

Sample 7 was a two-layer coating of FeAlN—AlN. The FeAlN was 800 nanometers thick. The AlN was 700 nanometers thick. The target material was 65 weight percent of Fe and 35 weight percent of Al.

Sample 8 was a control sample, an uncoated copper ring.

The raw imaging data contained a real part, and an imaginary part of the images. The square root of sum of (the [real part]²+the [imaginary part]²) is the magnitude. The arctangent of the imaginary part/real part is the phase. In FIG. 1, results are presented showing the magnitude of the images of the coated and uncoated samples.

Referring to FIG. 1, the original raw magnitude data is shown. It appears that sample number 2 is larger than control number eight.

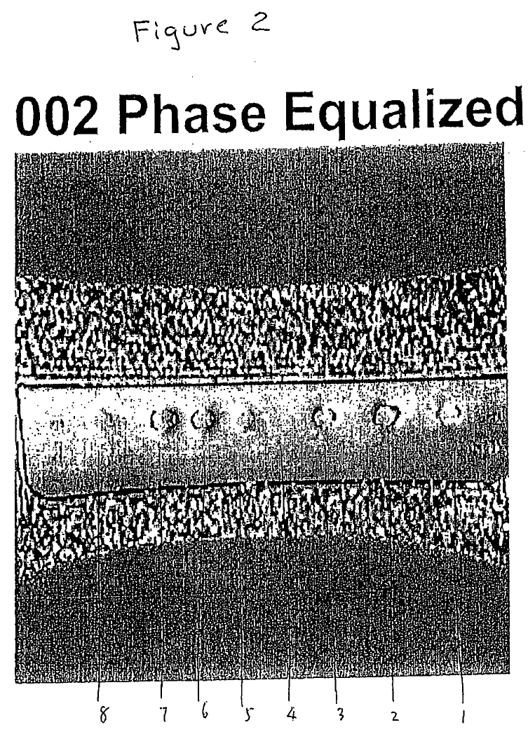

In FIG. 2, the phase image of the samples is presented. As will be apparent, sample number 4 is almost “invisible” in comparison to control sample number 8, and to the other samples.

As will be apparent, by varying the composition of the film components, and/or their thicknesses, and/or the layer stacking sequence(s), one may make the MRI image of the coating either more visible than the control, or less visible than the control.

Without wishing to be bound to any particular theory, applicants believe that the image produced is a function of the magnetic properties of the coating, the capacitative properties of the coating, and the relationship of the reactances that are caused by such properties. It is believed that the magnetic property causes inductive reactance; and it is believed that the insulating layer AlN causes capacitive reactance. When the inductive reactance is equal to the capacitive reactive, they cancel out if they are connected in series. Thus, the coating may be “stealthily tuned” so that the net reactance is substantially zero. The tuned or partially tuned sample will yield magnitude or phase images that are different from the control. See, e.g., sample 4 of FIG. 2.

Similarly, the coating may be “visibly tuned” so that the net reactance is great. This tuned sample will yield an enlarged imaging (see, e.g., sample 2 of FIG. 1).

Thus, e.g., if one wants to use the nanomagnetic particles as tracers, one can increase their magnitude in the manner done with sample 2. If, conversely, one wants to use these particles as MRI “stealth agents,” one may tune them in the manner indicated for sample 4 of the FIG. 2.

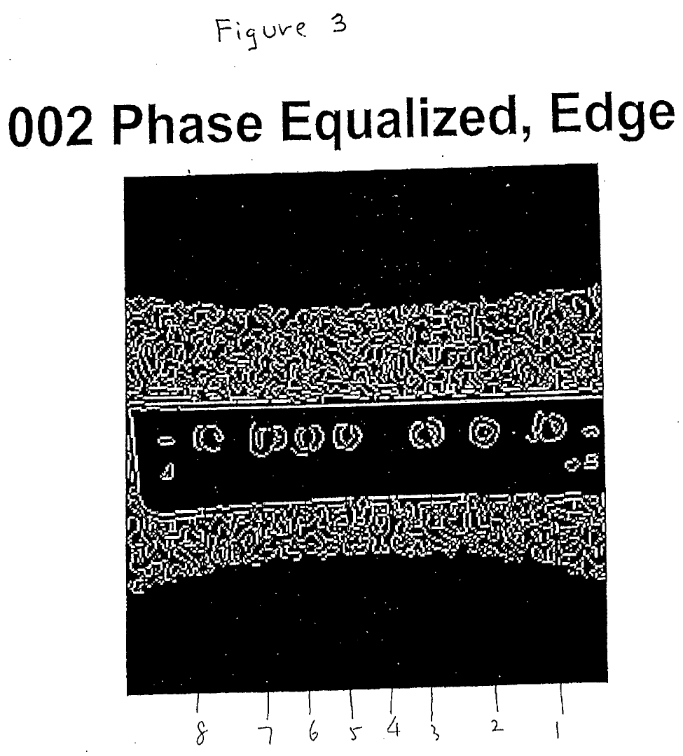

FIG. 3 illustrates the results obtained as a result of an edge-tracing mathematical calculation of FIG. 2. Note that, in this FIG. 3, sample 4 is substantially invisible.

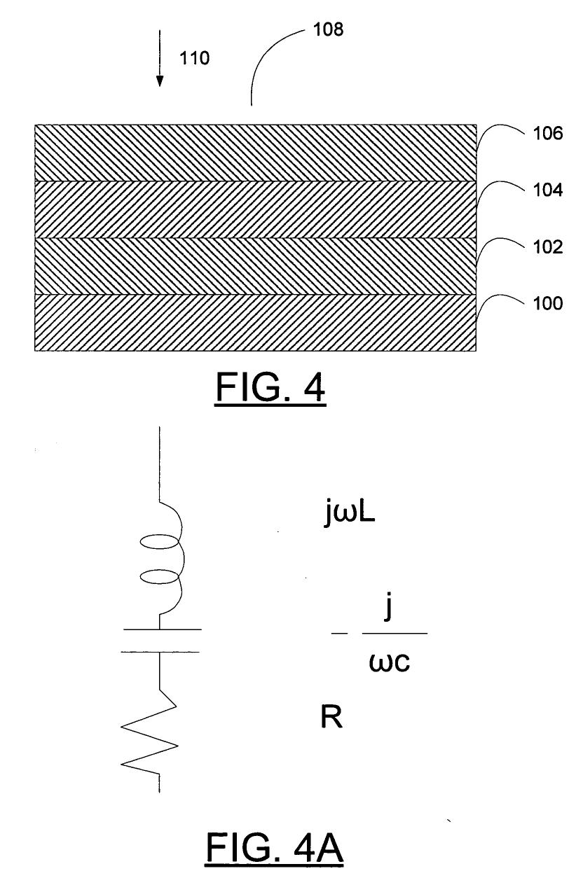

FIG. 4 is a schematic representation of conductor 100 coated with an insulating layer 102 and a nanomagnetic layer 104, and another insulating layer 106. The insulating layers may be comprised of nano-sized AlN particles, and the nanomagnetic layer 104 may be comprised of FeAlN nanoparticles.

When the assembly 108 is exposed to a high frequency magnetic field 110, eddy currents are minimized. In the absence of the insulating layers 102 and 106 and the nanomagnetic layer 104, substantial eddy currents will be induced in the conductor 100, thereby causing MRI image artifacts. By comparison, when the overall impedance of the AlN/FeAlN/AlN/conductor assembly is tuned to a non-reactive value, the eddy current generation, and the consequent production of image artifacts, is minimized.

FIG. 4A is a simplified presentation of the circuit that is believed to exist in assembly 108. The capacitance is created by the layers of the AlN insulating material. The inductance is created by FeAlN nanomagnetic material. The resistance is related to dissipative energy loss.

Referring to the circuit depicted in FIG. 4A, when the inductive reactance (ωL) is equal to the capacitive reactance (1/ωC), the overall reactance of the circuit depicted in FIG. 4A is at minimum for the series circuit depicted. With such minimal reactance, there will no phase shift, and no image artifacts. Reference also may be had sample 4 of FIG. 3, and also to sample 4 of FIG. 2, where there is minimal phase shift due to net reactance and minimal image artifacts.

Literature References Disclosing Conjugation of Magnetic Particles to Ligands

In addition to the patents and published patent applications discussed elsewhere in this specification, there are a substantial number of literature references disclosing methods for conjugating magnetic materials to ligands. Some of these are discussed below by way of illustration.

In an article by Laura G. Remsen et al., published in AJNR Am J. Neuroradiol 17:411-418, March 1996 (“MR of Carcinoma-Specific Monoclonal Antibody Conjugated to Moncrystalline Iron Oxide Nanoparticles”), a process is described in which tumor-specific monoclonal antibodies are conjugated to moncrystalline iron oxide nanoparticles (MIONS). This article also discloses that “Paramagnetic gadolinium chelates have been used clinically as magnetic resonance (MR) imaging agents . . . .”

In an article by Dagmar Hogemann et al. (“Improvement of MRI Probes to Allow Efficient Detection of Gene Expression”), published in Bioconjugate Chem. 2000, 11, 941-946, the authors discussed a process in which dextran coated MIONS were conjugated to transferring. This was done in an attempt to conduct “Real-time noninvasive imaging of gene expression in vivo . . . .”

In an article by Su Xu et al. (“Study . . . Using MION-461 Enhanced In Vivo MRI . . . ”), published in Journal of Neuroscience Research 52:549-558 (1998), the authors disclose that: “Gadolinium chelate-enhanced MRI has also been used . . . . However, the rapid clearance and short circulating half-life of gadolinium chelates have impaired the ability to correlate areas of BBB disruption to histopathology.” The authors also disclose that “ . . . recent work has used the monocrystaline iron oxide nanoparticle MION-46 . . . to demonstrate brain lesions following osmotic BBB disruption in rats . . . MION-46 has potential to improve focal lesion detection is small animals because it may increase the imaging contrast between the lesion and surrounding tissue. MION-46L has a longer circulating half-life, stronger spin-spin relaxivity . . . , and larger induced magnetic susceptibility compared to clinically used gadolinium chelates.”

In an article by S. Ozawa et l. (“What's new in imaging? . . . ”), published in 1: Recent Results Cancer Res. 2000; 155: 73-87, the authors disclose the preparation of superparamagnetic particles coated with monoclonal antibodies directed against epidermal growth factor receptors which are over-expressed in esophageal squamous cell carcinoma.

As those in the art are well aware, there are many other disclosures of processes in which magnetic material is conjugated with a recognition molecule, such as an antibody.

The Use of a Recognition Molecule/Magnetic Material Conjugate as an Assay

Biochemists have developed a process for detecting miniscule amounts of protein in a biochemical soup. This process is discussed at page 1827 of SCIENCE, VOL. 301, 26 September 2003. According to this article, the Biochemists conjugated 1-micrometer plastic spheres with magnetic iron cores to genetically engineered monoclonal antibodies. The conjugate was then added to the “biochemical soup,” and a magnetic field was then turned on to attract the magnetic particles to the side of a test tube.

In one embodiment, applicants' preferred nanomagnetic particles are used with this process.

In another embodiment, and further to the ability to modulate the magnetic signal characteristic of discrete populations of these particles; MRI analytical protocols such as magnitude mapping, phase mapping, and other techniques known to those skilled in the art; will permit clear distinction to be made between the populations, irrespective of the local concentration of the particles. In this manner, a “cocktail” of several populations of particles, each population having been chemically bound to a different recognition molecule, may be introduced into a living organism. Each population of particle will selectively bind to a desired cell or tissue type, and the ability of MRI analysis to differentiate between the magnetic properties of the particle populations will permit highly enhanced diagnosis of disease states such as infection or cancer.

One application of the resulting diagnostic capability is to differentiate between, for example, a cyst and a tumor in breast tissue, without biopsy. Another application is to make use of multiple cell-surface receptors to greatly increase selectivity and specificity in diagnosing infectious agents. Those skilled in the art will understand the wide applicability of this technique to MRI, thus bringing far greater utility to this imaging modality.

A Novel Coated Stent

This second part of the patent application relates to a coated stent. As is known to those skilled in the art, and as is disclosed in U.S. Pat. No. 5,968,091, “Transluminal prostheses are well known in the medical arts for implantation in blood vessels, bilary ducts, or other similar organs of the living body. These prostheses are commonly known as stents and are used to maintain, open, or dilate tubular structures or to support tubular structures that are being anastomosed” (see column 1 of this patent).

Coated stents are known to those skilled in the art. Thus, by way of illustration and not limitation, reference may be had to U.S. Pat. No. 5,779,729 (coated stent), U.S. Pat. No. 6,666,880 (method for securing a coated stent to a balloon catheter), U.S. Pat. No. 6,626,815 (stent with radioactive coating), U.S. Pat. Nos. 6,579,311, 6,364,903 (polymer coated stent), U.S. Pat. Nos. 6,335,384, 6,264,936 (stent coated with antimicrobial materials), U.S. Pat. No. 6,251,136 (method of layering a three-coated stent), U.S. Pat. No. 6,126,658 (radiation coating U.S. Pat. Nos. 5,993,374, 5,968,091, 5,897,911 (polymer-coated stent structure), and the like.

The stent of this patent application is also a coated stent. The coated stent comprises a coating that contains a layer of FeAlN nanomagnetic particles whose properties have been described elsewhere in this specification. In one embodiment, the coating is a multi-layer structure, being a two-layer coating of FeAlN—AlN. In one aspect of this embodiment, the FeAlN is 800 nanometers thick, and the AlN is 700 nanometers thick.

Regardless of whether the coating has one, two, or three layers of material, it always preferably has at least one layer of FeAlN material. The thickness of this FeAlN material preferably ranges between 100 to about 2,000 nanometers and, more preferably, from about 400 to about 1,000 nanometers.

The FeAlN material used in the coating preferably contains from about 1 to about 39 weight percent of Fe (Fe/Fel+Al), by total weight of Fe and Al. In one embodiment, the FelAlN material used is comprised of from about 10 to about 30 weight percent of Fe, by total weight of Fe and Al.

It is preferred that at least about 0.1 moles of nitrogen are present in the FeAlN material, by reference to the total number of moles of nitrogen, Fe, and Al present in the material. In one preferred embodiment, at least about 0.2 moles of nitrogen are present in the FeAlN material.

The FeAlN material may be coated on the exterior surface of the stent. Additionally, and/or alternatively, it may be coated on the interior surface of the stent.

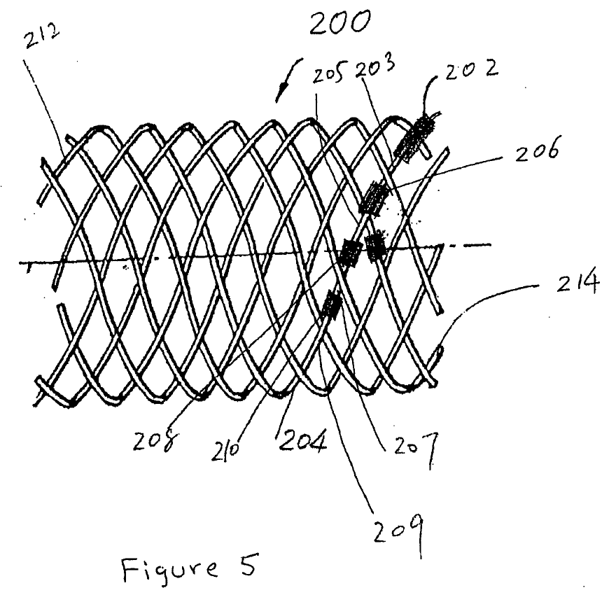

FIG. 5 is a perspective view of a coated stent 200. In the embodiment depicted, Stent 200 is comprised of an exterior coating 202. The exterior coating 202 may extend over the entire exterior surface of the stent 200 (not shown). Alternatively, some or all of stent 202 may have a portion of it intermittently coated with the FeAlN coating.

Thus, and by way of illustration, section 204 of stent 200 is comprised of coated sections 202, 206, 208, and 210 adjacent to uncoated sections 203, 205, 207, and, 209.

Without wishing to be bound to any theory, applicants believe that the uncoated sections 203 et seq. have a higher surface conductivity than the coated sections 202 et seq.

Thus, one may vary the amount of inductive reactance and/or capacitative reactance that is present during exposure to an MRI radio frequency field by several different means. One of such means is by using multiple coatings with differing electromagnetic properties to either maximize the difference in reactances (to maximize the image distortion), or to minimize the difference in reactances (to minimize the image distortion). Instead of using multiple coatings, and/or in addition to using multiple coatings, one may use intermittent coatings, as illustrated in FIG. 5 (see portion 202).

In another embodiment, one may use coatings both on the outside surface 212 of the stent and well as the inside surface 214. These exterior and/or interior coatings may have identical properties (such as thickness, conductivity, etc.), or they may have different properties.

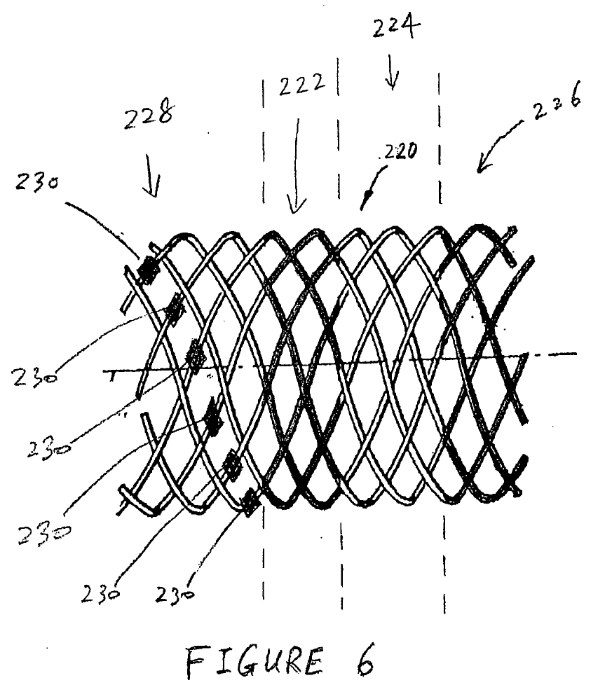

FIG. 6 is a perspective view of a stent 220 that has different sections with different properties. Referring to FIG. 6, it will b seen that section 222 is continuously coated with a coating comprising an FeAlN layer; section 224 is uncoated; section 226 is continuously coated; and section 228 is intermittently coated with a coating 230.

As will be apparent to those skilled in the art, by varying the thicknesses and/or the chemical properties and/or the layers and/or the coating patters of the stent and/or the stent portions, one may vary the response electromagnetic response of such stent to a radio frequency field.

Without wishing to be bound to any particular theory, applicants believe that the signals received during an MRI imaging process are comprised of a real part and an imaginary part of the image (see page 56 of this specification). The phase is the arctangent of the imaginary part/real part. It is believed that, in addition to its magnitude, the phase content is important in the visualization of the an object within the stent.

It is important to be able to visualize an object within a stent to determine, e.g., whether re-growth of plaque has occurred after the implantation of the stent. By utilizing the process described in the next portion of this specification, one can readily visualize such re-growth.

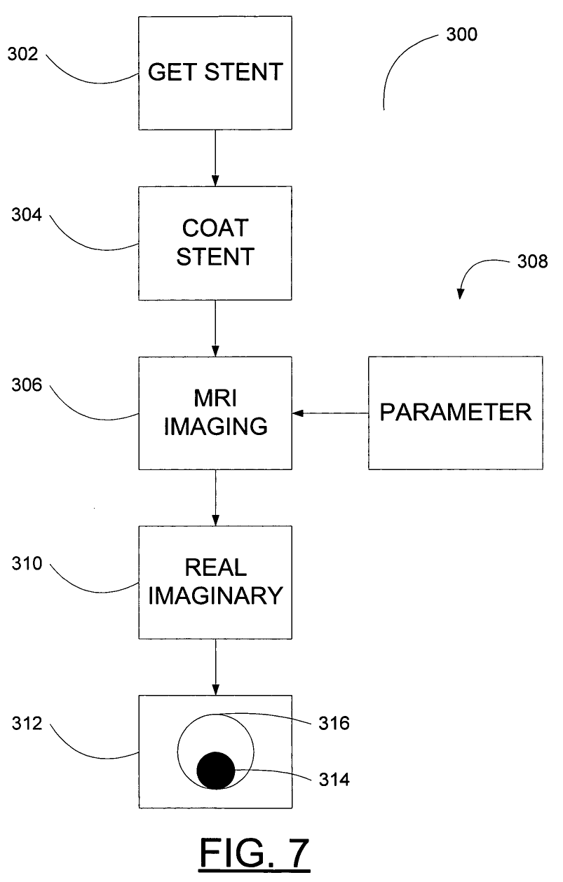

FIG. 7 is a flow diagram of a process 300 for visualizing the material inside of a stent. Referring to FIG. 7, and in step 302 thereof, a conventional stent is obtained. One may use one or more of the prior art stents such as, e.g., those disclosed in U.S. Pat. No. 5,653,727 (intravascular stent), U.S. Pat. No. 6,332,892 (medical device with one or more helical coils), U.S. Pat. No. 5,941,869 (method and apparatus for controlled removal of stenotic material from stents), U.S. Pat. No. 6,355,058 (stent with radio-opaque coating consisting of particles in a binder), U.S. Pat. No. 6,656,219 (intravascular stent), and the like. The entire disclosure of each of these United States patents is hereby incorporated by reference into this specification.

In one embodiment, the stent is comprised of at least about 50 weight percent of a metal or metal alloy such as, e.g., copper, stainless steel, nickel, tantalum, etc. These metal and/or metal alloy materials are frequently used to obtain a desired degree of flexibility with mechanical strength to the stent. However, the use of such metal or metal-containing materials in a mesh and/or coil configuration (see FIGS. 5 and 6) creates a structure with conducting loops that, in the presence of an high frequency alternating current field, causes large eddy currents to flow. These eddy currents, in turn, create image artifacts and prevent visualization of materials within the stent. The use of the coatings of this invention helps minimize these eddy currents.

Referring again to FIG. 7, and in step 304 thereof, the stent is coated with at least one layer of FeAlN material. The coating may optionally contain other layers of material, such, e.g., AlN. The purpose of this coating is to reduce the production of eddy currents when the stent is subjected to an MRI radio-frequency field.

In step 306 of the process, the coated stent is disposed within a phantom solution and subjected to the MRI fields normally present during the MRI imaging. The phantom solution used is chosen to simulate the bodily fluid of human beings. In one embodiment, and by way of illustration, vegetable oil is used.

In step of 308 of the process, the various parameters of the MRI imaging system are varied to determine how the coated stent responds under different conditions. Thus, e.g., one may utilize the Asymmetric Spin Echo for Bₒ parameter, and the Spin Echo with changes in the Transmit Gain (TG) for B₁. As will be apparent, this step 308 will tend to demonstrate the extent to which an object on the inside of the stent may be visualized under various conditions.

In one embodiment, the Transmit Gain (TG) of the system is from about 110 to about 200. In one aspect of this embodiment, the TG is varied to from about 140 to about 180. In another embodiment, the TG is varied from about 150 to about 175.

For any desired visualization result, and for any particular coated stent structure, there will be optimal MRI parameters that will facilitate obtaining such result.

In step 310, the “real” and “imaginary” results obtained are subjected to image processing. One may use the image processing techniques known to those skilled in the art. Reference may be had, e.g., to U.S. Pat. No. 5,878,165 (method for extracting object images), U.S. Pat. Nos. 5,751,831, 6,073,041 (physiological corrections in functional magnetic resonance imaging), U.S. Pat. No. 6,426,994 (image processing method), U.S. Pat. No. 6,621,433 (adaptive dynamic receiver for MRI), U.S. Pat. No. 6,374,135 (system for indicating the position of a surgical probe within a head on an image of the head), U.S. Pat. No. 6,118,845 (system and methods for the reduction and elimination of image artifacts), U.S. Pat. No. 6,584,210 (digital watermark image processing method), U.S. Pat. No. 5,003,979 (system for the noninvasive identification and display of breast lesions), U.S. Pat. No. 6,556,720 (method and apparatus for enhancing and correcting digital images), U.S. Pat. No. 6,597,935 (method for harmonic phase magnetic resonance imaging) and the like. The entire disclosure of each of these United States patents is hereby incorporated by reference into this specification.

In one embodiment, the image processing involves the step of equalization. As is known to those skilled in the art, in this step the number of pixels is equalized as a function of the gray levels in the image. Reference may be had, e.g., to U.S. Pat. No. 4,991,092 (image processor for enhancing contrast between subregions of a region of interest), U.S. Pat. No. 5,005,578 (three-dimensional magnetic resonance image distortion correction method), U.S. Pat. No. 6,424,730 (medical image enhancement method), U.S. Pat. No. 5,150,421 (system for automated transformation of gray level of image), U.S. Pat. No. 5,681,112 (image enhancement), U.S. Pat. No. 6,556,720 (method and apparatus for enhancing and correcting digital images), and the like. The entire disclosure of each of these United States patents is hereby incorporated by reference into this specification.

In step 312, the visual results produced by steps 302 et seq. are displayed. Referring to step 312, it will be seen that an area of re-growth 314 is shown within the stent 316.

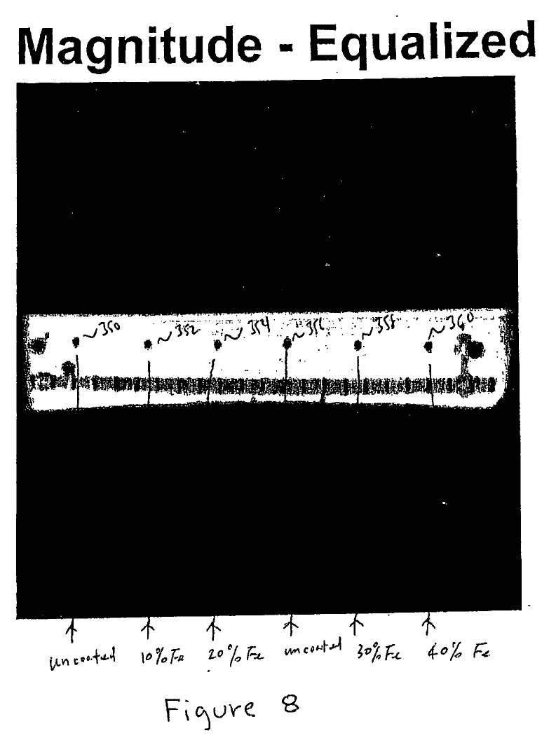

FIG. 8 is an imaging produced from a number of different coatings on a series of copper stents. Stent 350 was uncoated. Stent 352 was coated with a layer of FeAlN, 800 nanometers thick, and a layer of AlN, 700 nanometers thick; in stent 352, the concentration of Fe (by weight of Fe+Al) was 10 percent. Stent 354 was similar to stent 352, but the concentration of Fe was 20 percent. Stent 356 was similar to stent 350, being uncoated. Stent 358 was similar to stent 354, but it contained 30 percent of Fe. Stent 360 contained 40 percent of Fe. As will be apparent, the equalization of the magnitude did not readily facilitate the visualization of objects within the stent.

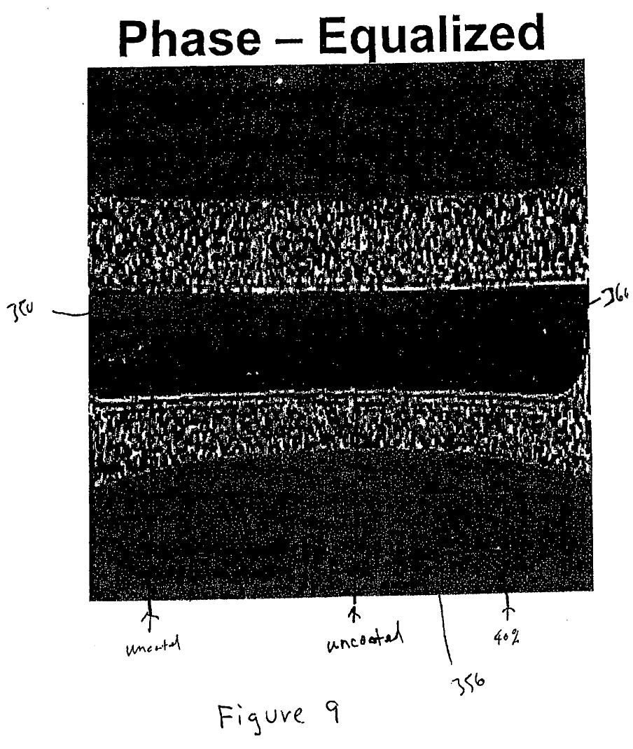

FIG. 9 is an imaging produced from the stents of FIG. 8, wherein the phase was equalized. It will be seen that, with this process, stents 352, 354, and 358 were rendered “invisible,” i.e., the stent was removed from the image.

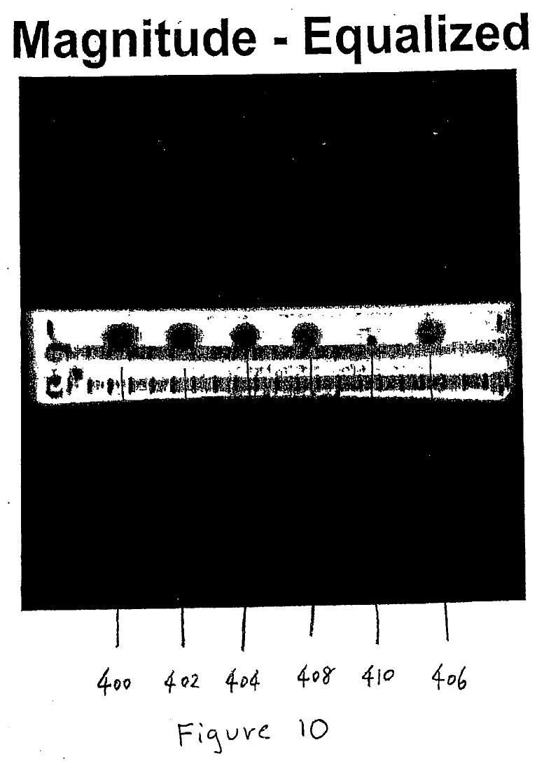

FIG. 10 is an imaging produced from the series of copper rings that were used to simulate the stents of FIG. 8, wherein the magnitude only is equalized. In the experiments of FIG. 10, a nylon bolt was disposed within each such copper ring.

Rings 400, 402, 404, and 406 were not coated. Copper ring 408 was coated with an intermittent coating (see FIGS. 5 and 6 and sections 202, 222, 226, and 230). The coating used was similar to that of stent 352, but it differed in that it contained alternating coated areas (about 5 millimeters wide) and uncoated areas (about 2-3 millimeters wide).

Referring again to FIG. 10, ring 410 was uncoated but cut so that it did not form a continuous conductive loop. As will apparent, the nylon bolt was best visualized in ring 410. However, and as also will be apparent, a cut ring does not afford enough mechanical strength to be used as a stent.

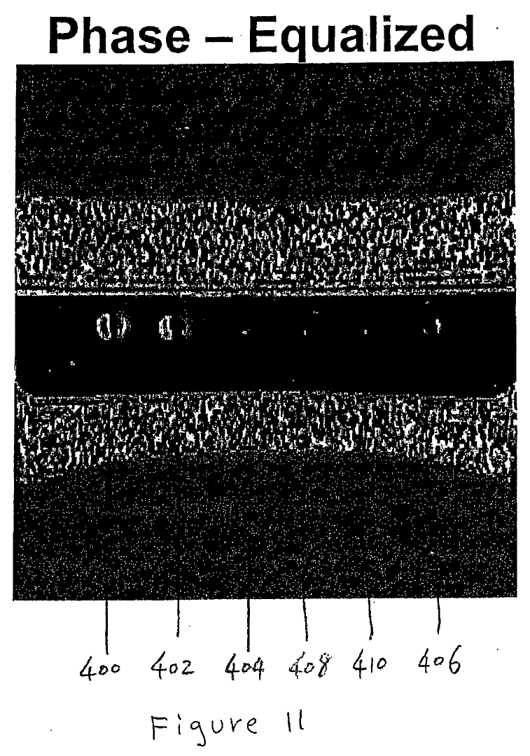

FIG. 11 better shows the differences between the uncoated integral rings, and the coated integral rings. In FIG. 11, however, the phase has been equalized.

Referring to FIG. 11, it will be seen that the uncoated samples 400, 402, and 406 all depict the nylon bolt in distorted fashion. The uncoated sample 404 also had some distortion.

By comparison, the coated sample 408 provided a clear, undistorted visualization of the nylon bolt that was substantially as good as the cut ring sample 410. One was thus able to visualize the nylon bolt in sample 408 without destroying the structural integrity of the ring.

The foregoing description details the embodiments most preferred by the inventors. Variations to the foregoing embodiments will be readily apparent to those skilled in the relevant art. Therefore the scope of the invention should be measured by the appended claims.

We claim:

1. A MRI contrast enhancing material comprised of nanomagnetic particles having a particle size less than 100 nanometers, a coercive magnetic force of from about 1 to about 200 Oersteds, a remnant magnetization of from about 10 to about 10,000 Gauss, and a saturation magnetization of from about 100 to about 24,000 Gauss.

2. The MRI contrast enhancing material as recited in claim 1, wherein said nanomagnetic particles have a particle size distribution such that at least about 50 percent of said nanomagnetic particles have an average size of from about 3 to about 10 nanometers.

3. The MRI contrast enhancing material as recited in claim 1, wherein said nanomagnetic particles have a particle size distribution such that at least about 60 percent of said nanomagnetic particles have an average size of from about 6 to about 10 nanometers.

4. The MRI contrast enhancing material as recited in claim 1, wherein said nanomagnetic particles have a particle size distribution such that at least about 80 percent of said nanomagnetic particles have an average size of from about 6 to about 10 nanometers.

5. The MRI contrast enhancing material as recited in claim 1, wherein said nanomagnetic particles have a coercive force of from about 10 to about 120 Oersteds.

6. The MRI contrast enhancing material as recited in claim 5, wherein said nanomagnetic particles have a coercive force of from about 20 to about 110 Oersteds.

7. The MRI contrast enhancing material as recited in claim 1, wherein said nanomagnetic particles have a remnant magnetization of from about 1,000 to about 8,000 Gauss.

8. The MRI contrast enhancing material as recited in claim 7, wherein said nanomagnetic particles have a remnant magnetization of from about 2,000 to about 7,500 Gauss.

9. The MRI contrast enhancing material as recited in claim 1, wherein said nanomagnetic particles have a saturation magnetization of from about 10,000 to about 21,000.

10. The MRI contrast enhancing material as recited in claim 1, wherein said nanomagnetic particles have a relative magnetic permeability of from about 1 to about 500,000.

11. The MRI contrast enhancing material as recited in claim 10, wherein said nanomagnetic particles have a relative magnetic permeability of from about 1.5 to about 260,000.

12. The MRI contrast enhancing material as recited in claim 11, wherein said nanomagnetic particles have a relative magnetic permeability of from about 1.5 to about 2000.

13. The MRI contrast enhancing material as recited in claim 1, wherein said nanomagnetic particles are comprised of at least one of three distinct elemental moieties, moiety A, moiety B, and moiety C.

14. The MRI contrast enhancing material as recited in claim 13, wherein moiety A is magnetic and is chosen from the group consisting of iron, nickel, cobalt, alloys thereof, and mixtures thereof, moiety B is non-magnetic and is chosen from the group consisting of silicon, aluminum, boron, platinum, tantalum, palladium, yttrium, zirconium, titanium, calcium, berylium, barium, silver, gold, indium, lead, tin, antimony, germanium, gallium, tungsten, bismuth, strontium, magnesium, and zinc, and moiety C is chosen from the group consisting of oxygen, nitrogen, carbon, fluorine, chlorine, hydrogen, helium, neon, argon, krypton, and xenon.

15. The MRI contrast enhancing material as recited in claim 14, wherein the molar ratio of moiety A to (moiety A+moiety C) is from about 1 to about 99 mole percent.

16. The MRI contrast enhancing material as recited in claim 15, wherein the molar ratio of moiety A to (moiety A+moiety C) is from about 10 to about 90 mole percent.

17. The MRI contrast enhancing material as recited in claim 16, wherein the molar ratio of moiety A to (moiety A+moiety C) is from about 40 to about 60 mole percent.

18. The MRI contrast enhancing material as recited in claim 14, wherein the molar ratio of moiety A to (moiety A+moiety B+moiety C) is from about 1 to about 99 mole percent.

19. The MRI contrast enhancing material as recited in claim 18, wherein the molar ratio of moiety A to (moiety A+moiety B+moiety C) is from about 10 to about 90 mole percent.

20. The MRI contrast enhancing material as recited in claim 19, wherein the molar ratio of moiety A to (moiety A+moiety B+moiety C) is from about 30 to about 60 mole percent.

21. The MRI contrast enhancing material as recited in claim 14, wherein the molar ratio of moiety B to (moiety A+moiety B+moiety C) is from about 1 to about 99 mole percent.

22. The MRI contrast enhancing material as recited in claim 21, wherein the molar ratio of moiety B to (moiety A+moiety B+moiety C) is from about 10 to about 40 mole percent.

23. The MRI contrast enhancing material as recited in claim 14, wherein the molar ratio of moiety C to (moiety A+moiety B+moiety C) is from about 1 to about 99 mole percent.

24. The MRI contrast enhancing material as recited in claim 23, wherein the molar ratio of moiety C to (moiety A+moiety B+moiety C) is from about 10 to about 50 mole percent.

25. The MRI contrast enhancing material as recited in claim 14, wherein said moiety A is iron and said moiety B is aluminum.

26. The MRI contrast enhancing material as recited in claim 25, wherein said moiety C is nitrogen.

27. A stent comprising: a base substrate having a surface, said surface having an exterior section contiguous with an interior section; a plurality of layered coatings disposed on at least a portion of said surface, at least one of said plurality of layered coatings being comprised of a nanomagnetic material, said nanomagnetic material comprised of nanomagnetic particles having a particle size less than 100 nanometers, a coercive magnetic force of from about 1 to about 200 Oersteds, a remnant magnetization of from about 10 to about 10,000 Gauss, and a saturation magnetization of from about 100 to about 24,000 Gauss.

28. The stent as recited in claim 27, wherein said nanomagnetic particles have a particle size distribution such that at least about 50 percent of said nanomagnetic particles have an average size of from about 3 to about 10 nanometers.

29. The stent as recited in claim 27, wherein said nanomagnetic particles have a particle size distribution such that at least about 60 percent of said nanomagnetic particles have an average size of from about 6 to about 10 nanometers.

30. The stent as recited in claim 27, wherein said nanomagnetic particles have a particle size distribution such that at least about 80 percent of said nanomagnetic particles have an average size of from about 6 to about 10 nanometers.

31. The stent as recited in claim 27, wherein said nanomagnetic particles have a coercive force of from about 10 to about 120 Oersteds.

32. The stent as recited in claim 31, wherein said nanomagnetic particles have a coercive force of from about 20 to about 110 Oersteds.

33. The stent as recited in claim 27, wherein said nanomagnetic particles have a remnant magnetization of from about 1,000 to about 8,000 Gauss.

34. The stent as recited in claim 33, wherein said nanomagnetic particles have a remnant magnetization of from about 2,000 to about 7,500 Gauss.

35. The stent as recited in claim 27, wherein said nanomagnetic particles have a saturation magnetization of from about 10,000 to about 21,000.

36. The stent as recited in claim 27, wherein said nanomagnetic particles have a relative magnetic permeability of from about 1 to about 500,000.

37. The stent as recited in claim 36, wherein said nanomagnetic particles have a relative magnetic permeability of from about 1.5 to about 260,000.

38. The stent as recited in claim 37, wherein said nanomagnetic particles have a relative magnetic permeability of from about 1.5 to about 2000.

39. The stent as recited in claim 27, wherein said nanomagnetic particles are comprised of at least one of three distinct elemental moieties, moiety A, moiety B, and moiety C.

40. The stent as recited in claim 39, wherein moiety A is magnetic and is chosen from the group consisting of iron, nickel, cobalt, alloys thereof, and mixtures thereof, moiety B is non-magnetic and is chosen from the group consisting of silicon, aluminum, boron, platinum, tantalum, palladium, yttrium, zirconium, titanium, calcium, berylium, barium, silver, gold, indium, lead, tin, antimony, germanium, gallium, tungsten, bismuth, strontium, magnesium, and zinc, and moiety C is chosen from the group consisting of oxygen, nitrogen, carbon, fluorine, chlorine, hydrogen, helium, neon, argon, krypton, and xenon.

41. The stent as recited in claim 40, wherein the molar ratio of moiety A to (moiety A+moiety C) is from about 1 to about 99 mole percent.

42. The stent as recited in claim 41, wherein the molar ratio of moiety A to (moiety A+moiety C) is from about 10 to about 90 mole percent.

43. The stent as recited in claim 42, wherein the molar ratio of moiety A to (moiety A+moiety C) is from about 40 to about 60 mole percent.

44. The stent as recited in claim 40, wherein the molar ratio of moiety A to (moiety A+moiety B+moiety C) is from about 1 to about 99 mole percent.

45. The stent as recited in claim 44, wherein the molar ratio of moiety A to (moiety A+moiety B+moiety C) is from about 10 to about 90 mole percent.

46. The stent as recited in claim 45, wherein the molar ratio of moiety A to (moiety A+moiety B+moiety C) is from about 30 to about 60 mole percent.

47. The stent as recited in claim 40, wherein the molar ratio of moiety B to (moiety A+moiety B+moiety C) is from about 1 to about 99 mole percent.

48. The stent as recited in claim 47, wherein the molar ratio of moiety B to (moiety A+moiety B+moiety C) is from about 10 to about 40 mole percent.

49. The stent as recited in claim 40, wherein the molar ratio of moiety C to (moiety A+moiety B+moiety C) is from about 1 to about 99 mole percent.

50. The stent as recited in claim 49, wherein the molar ratio of moiety C to (moiety A+moiety B+moiety C) is from about 10 to about 50 mole percent.

51. The stent as recited in claim 40, wherein said plurality of layered coatings comprises a first layer coated with a second layer, wherein in said first layer said moiety A is absent, said moiety B is aluminum, and said moiety C is nitrogen, wherein in said second layer said moiety A is iron, said moiety B aluminum, and said moiety C is nitrogen.

52. The stent as recited in claim 51, wherein said first layer has a thickness of from about 400 to about 700 nanometers and said second layer has a thickness of from about 100 to about 2000 nanometers.

53. The stent as recited in claim 52, wherein said second layer has a thickness of from about 400 to about 1000 nanometers.

54. The stent as recited in claim 51, wherein said second layer is comprised of from about 1 to about 39 weight percent of iron, by total weight of iron and aluminum.

55. The stent as recited in claim 54, wherein said second layer is further comprised of 0.1 moles of nitrogen.

56. The stent as recited in claim 54, wherein said second layer is further comprised of 0.2 moles of nitrogen.

57. The stent as recited in claim 54, wherein said second layer is comprised of from about 10 to about 30 weight percent of iron, by total weight of iron and aluminum.

58. The stent as recited in claim 57, wherein said second layer is further comprised of 0.1 moles of nitrogen.

59. The stent as recited in claim 57, wherein said second layer is further comprised of 0.2 moles of nitrogen.

60. The stent as recited in claim 40, wherein said plurality of layered coatings comprises a first layer coated with a second layer coated with a third layer, wherein in said first layer and in said third layer said moiety A is absent, said moiety B is aluminum, and said moiety C is nitrogen, wherein in said second layer said moiety A is iron, said moiety B aluminum, and said moiety C is nitrogen.

61. The stent as recited in claim 60, wherein said first layer and said third layer have a thickness of from about 400 to about 700 nanometers and said second layer has a thickness of from about 100 to about 2000 nanometers.

62. The stent as recited in claim 61, wherein said second has a thickness of from about 400 to about 1000 nanometers.

63. The stent as recited in claim 60, wherein said second layer is comprised of from about 1 to about 39 weight percent of iron, by total weight of iron and aluminum.

64. The stent as recited in claim 63, wherein said second layer is further comprised of 0.1 moles of nitrogen.

65. The stent as recited in claim 63, wherein said second layer is further comprised of 0.2 moles of nitrogen.

66. The stent as recited in claim 60, wherein said second layer is comprised of from about 10 to about 30 weight percent of iron, by total weight of iron and aluminum.

67. The stent as recited in claim 66, wherein said second layer is further comprised of 0.1 moles of nitrogen.

68. The stent as recited in claim 66, wherein said second layer is further comprised of 0.2 moles of nitrogen.

69. The stent as recited in claim 27, wherein said plurality of layered coatings is disposed only on portions of said exterior section of said surface.

70. The stent as recited in claim 27, wherein said plurality of layered coatings is disposed continuously only on said exterior section of said surface.

71. The stent as recited in claim 27, wherein said plurality of layered coatings is disposed only on portions of said interior section of said surface.

72. The stent as recited in claim 27, wherein said plurality of layered coatings is disposed continuously only on said interior section of said surface.

73. The stent as recited in claim 27, wherein said plurality of layered coatings is disposed continuously on said surface.
